(19) United States
(12) Patent Application Publication
(10) Pub. No.: US 2010/0063049 A1
(43) Pub. Date: Mar. 11, 2010

(54) 2-CARBOCYCLOAMINO-4-IMIDAZOLYLPYRIMIDINES AS AGENTS FOR THE INHBITION OF CELL PROLIFERATION

(76) Inventor: Clifford JONES, Macclesfield, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/302,364

(22) PCT Filed: May 24, 2007
(86) PCT No.: PCT/GB2007/001939

§ 371 Date: Nov. 25, 2008

Related U.S. Application Data
(60) Provisional application No. 60/803,279, filed on May 26, 2006.
(60) Provisional application No. 60/869,043, filed on Dec. 7, 2006.

(51) Int. Cl. A61K 31/506; C07D 403/04; C07D 413/14; A61K 31/5377; A61P 35/00; A61P 35/02

(52) U.S. Cl. 514/235.8; 544/331; 544/122; 514/275

(57) ABSTRACT

Compounds of formula (I):

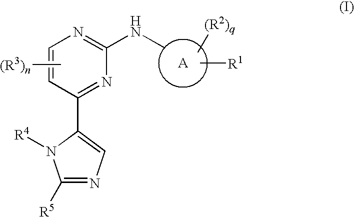

which possess cell-cycle inhibitory activity are described.

The invention relates to pyrimidine derivatives, or pharmaceutically acceptable salts or in vivo hydrolysable esters thereof, which possess cell-cycle inhibitory activity and are accordingly useful for their anti-cell-proliferation (such as anti-cancer) activity and are therefore useful in methods of treatment of the human or animal body. The invention also relates to processes for the manufacture of said pyrimidine derivatives, to pharmaceutical compositions containing them and to their use in the manufacture of medicaments of use in the production of an anti-cell-proliferation effect in a warm-blooded animal such as man.

The cell cycle is fundamental to the survival, regulation and proliferation of cells and is highly regulated to ensure that each step progresses in a timely and orderly manner. The progression of cells through the cell cycle arises from the sequential activation and de-activation of several members of the cyclin-dependent kinase (CDK) family. The activation of CDKs is dependent on their interaction with a family of intracellular proteins called cyclins. Cyclins bind to CDKs and this association is essential for CDK activity within the cell. Different cyclins are expressed and degraded at different points in the cell cycle to ensure that activation and inactivation of CDKs occurs in the correct order for progression through the cell cycle.

Moreover, CDKs appear to be downstream of a number of oncogene signalling pathways. Deregulation of CDK activity by upregulation of cyclins and/or deletion of endogenous inhibitors appears to be an important axis between mitogenic signalling pathways and proliferation of tumour cells.

Accordingly it has been recognised that an inhibitor of cell cycle kinases, particularly inhibitors of CDK1, CDK2, CDK4 and CDK6 (which operate at the G2/M, G1/S—S-G2/M and G1-S phases respectively) should be of value as an active inhibitor of cell proliferation, such as growth of mammalian cancer cells.

Tumour cells are also thought to be highly dependent on the continual transcriptional activity of RNA polymerase II to maintain appropriate levels of anti-apoptotic proteins and ensure tumour cell survival. CDK1, CDK7, CDK8 and CDK9 in particular are known to regulate the activity of RNA polymerase II through phosphorylation of the C-terminal domain of the protein. Thus, the inhibition of RNA polymerase II activity through inhibitors of these CDKs may contribute to a pro-apoptotic effect in tumour cells.

The inhibition of cell cycle kinases is expected to be of value in the treatment of disease states associated with aberrant cell cycles and cell proliferation such as cancers (solid tumours and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

WO 02/20512, WO 03/076435, WO 03/076436, WO 03/076434, WO 03/076433 and WO 04/101549 describe certain 2-anilino-4-imidazolylpyrimidine derivatives that inhibit the effect of cell cycle kinases. The present invention is based on the discovery that a novel group of non-anilino pyrimidines inhibit the effects of CDK2, and thus possess anti-cell-proliferation properties.

Accordingly, the present invention provides a compound of formula (I):

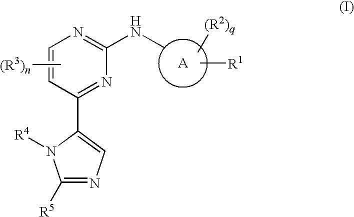

wherein:

Ring A is a 5-7 membered saturated carbocyclic ring wherein 2 atoms of Ring A may optionally be connected by a bridge;

R¹ is selected from carboxy, amino, sulphamoyl, sulphamoylamino, carbamoyl, a group —R⁶-R⁷ or a nitrogen linked 4-7 membered saturated ring which optionally contains an additional nitrogen, oxygen or sulphur atom; wherein said ring may be optionally substituted on carbon by one or more R⁸; and wherein if said ring contains an additional nitrogen atom that nitrogen may be optionally substituted by R⁹;

R² is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆ alkanoylamino, N—(C₁₋₆alkyl)carbamoyl, N,N—(C₁₋₆alkyl)₂carbamoyl, C₁₋₆ alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;

q is 0-4; wherein the values of R² may be the same or different;

R³ is selected from halo, cyano or amino;

n is 0 to 2, wherein the values of R³ may be the same or different;

R⁴ is selected from ethyl, propyl, isopropyl, butyl, iso-butyl, sec-butyl, t-butyl, cyclopropyl, cyclopropylmethyl, 1-cyclopropylethyl, cyclobutylmethyl, cyclopentyl or cyclobutyl; wherein R⁴ may be optionally substituted on carbon by one or more R¹⁴;

R⁵ is selected from methyl, ethyl, isopropyl, fluoromethyl, difluoromethyl, trifluoromethyl, methoxymethyl, cyclopropylmethyl or cyclopropyl;

R⁶ is selected from —O—, —N(R¹⁵)—, —C(O)—, —C(O)O—, —N(R¹⁶)C(O)—, —C(O)N(R¹⁷)—, —N(R¹⁸)C(O)O—, —N(R¹⁹)C(O)N(R²⁰)—, —S(O)ᵣ—, —OC(O)N(R²¹)SO₂—, —N(R²²)SO₂N(R²³)—, —SO₂N(R²⁴)—, —N(R²⁵)SO₂—, —C(O)N(R³⁹)SO₂— or —SO₂N(R⁴⁰)C(O)—; wherein R¹⁵, R¹⁶, R¹⁷, R¹⁸, R¹⁹, R²⁰, R²¹, R²², R²³, R²⁴, R²⁵, R³⁹ and R⁴⁰ are independently hydrogen or C₁₋₆alkyl optionally substituted by one or more R²⁶ and r is 0-2;

R⁷ is selected from C₁₋₆alkyl, C₂₋₆alkenyl, C₂₋₆alkynyl, carbocyclyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;

R⁸, R¹², R²⁶ and R²⁷ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkanoyl, C₁₋₆alkanoyloxy, N—(C₁₋₆alkyl)amino, N,N—(C₁₋₆alkyl)₂-amino, C₁₋₆ alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆ alkyl)sulphamoyl, N,N—(C₁₋₆alkyl)₂sulphamoyl, C₁₋₆alkylsulphonylamino, carbocyclyl-R²⁹— or heterocyclyl-R³⁰—; wherein R⁸, R¹², R²⁶ and R²⁷ independently of each other may be optionally substituted on carbon by one or more R³¹; and wherein if said heterocyclyl contains an —NH— moiety that nitrogen may be optionally substituted by a group selected from R³²;
R⁹, R¹³, R²⁸ and R³² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹³, R²⁸ and R³² independently of each other may be optionally substituted on carbon by one or more R³³; and

R¹⁰, R¹¹, R²⁹ and R³⁰ are independently selected from a direct bond, —O—, —N(R³⁴)—, —C(O)—, —N(R³⁵)C(O)—, —C(O)N(R³⁶)—, —S(O)ₛ—, —S(O)₂N(R³⁷)— or —N(R³⁸)SO₂—; wherein R³⁴, R³⁵, R³⁶, R³⁷ and R³⁸ are independently selected from hydrogen or C₁₋₆alkyl and s is 0-2;

R¹⁴ is selected from halo, nitro, cyano, hydroxy, amino, carboxy, carbamoyl, mercapto, sulphamoyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O), wherein a is 0 to 2, C₁₋₆alkoxycarbonyl, N—(C₁₋₆alkyl)sulphamoyl, N,N—(C₁₋₆alkyl)₂sulphamoyl and C₁₋₆alkylsulphonylamino;

R³¹ and R³³ are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt or an in vivo hydrolysable ester thereof.

According to a further feature of the present invention there is provided a compound of formula (I) wherein:

Ring A is a 5-7 membered saturated carbocyclic ring wherein 2 atoms of Ring A may optionally be connected by a bridge;

R¹ is selected from amino, sulphamoyl, sulphamoylamino, carbamoyl, a group —R⁶-R⁷ or a nitrogen linked 4-7 membered saturated ring which optionally contains an additional nitrogen, oxygen or sulphur atom; wherein said ring may be optionally substituted on carbon by one or more R⁸; and wherein if said ring contains an additional nitrogen atom that nitrogen may be optionally substituted by R⁹;

R² is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;

q is 0-4; wherein the values of R² may be the same or different;

R³ is selected from halo, cyano or amino;

n is 0 to 2, wherein the values of R³ may be the same or different;

R⁴ is selected from ethyl, propyl, isopropyl, butyl, iso-butyl, sec-butyl, t-butyl, cyclopropyl, cyclopropylmethyl, 1-cyclopropylethyl, cyclobutylmethyl, cyclopentyl or cyclobutyl; wherein R⁴ may be optionally substituted on carbon by one or more R¹⁴;

R⁵ is selected from methyl, ethyl, isopropyl, fluoromethyl, difluoromethyl, trifluoromethyl, methoxymethyl, cyclopropylmethyl or cyclopropyl;

R⁶ is selected from —O—, —N(R¹⁵)—, —C(O)—, —N(R¹⁶)C(O)—, —C(O)N(R¹⁷)—, —N(R¹⁸)C(O)O—, —N(R¹⁹)C(O)N(R²⁰)—, —S(O)ᵣ—, —OC(O)N(R²¹)SO₂—, —N(R²²)SO₂N(R²³)—, —SO₂N(R²⁴)— or —N(R²⁵)SO₂—; wherein R¹⁵, R¹⁶, R¹⁷, R¹⁸, R¹⁹, R²⁰, R²¹, R²², R²³, R²⁴ and R²⁵ are independently hydrogen or C₁₋₆alkyl optionally substituted by one or more R²⁶ and r is 0-2;

R⁷ is selected from C₁₋₆alkyl, C₂₋₆alkenyl, C₂₋₆alkynyl, carbocyclyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;

R⁸, R¹², R²⁶ and R²⁷ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²⁹— or heterocyclyl-R³⁰—; wherein R⁸, R¹², R²⁶ and R²⁷ independently of each other may be optionally substituted on carbon by one or more R³¹; and wherein if said heterocyclyl contains an —NH— moiety that nitrogen may be optionally substituted by a group selected from R³²;

R⁹, R¹³, R²⁸ and R³² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹³, R²⁸ and R³² independently of each other may be optionally substituted on carbon by one or more R³³; and

R¹⁰, R¹¹, R²⁹ and R³⁰ are independently selected from a direct bond, —O—, —N(R³⁴)—, —C(O)—, —N(R³⁵)C(O)—, —C(O)N(R³⁶)—, —S(O)ₛ—, —SO₂N(R³⁷)— or —N(R³⁸)SO₂—; wherein R³⁴, R³⁵, R³⁶, R³⁷ and R³⁸ are independently selected from hydrogen or C₁₋₆alkyl and s is 0-2;

R¹⁴ is selected from halo, nitro, cyano, hydroxy, amino, carboxy, carbamoyl, mercapto, sulphamoyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl and C₁₋₆alkylsulphonylamino;

R³¹ and R³³ are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt or an in vivo hydrolysable ester thereof.

In this specification the term “alkyl” includes both straight and branched chain alkyl groups but references to individual alkyl groups such as “propyl” are specific for the straight chain version only. For example, “C₁₋₆alkyl” includes methyl, ethyl, propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

Ring A is a “5-7 membered saturated carbocyclic ring”. A “5-7 membered saturated carbocyclic ring” is a saturated carbon ring that contains 5, 6 or 7 atoms; wherein a —CH₂— group can optionally be replaced by a —C(O)—. Particular examples of a “5-7 membered saturated carbocyclic ring” are cyclopentyl, cyclohexyl, cycloheptyl, 2-oxocyclopentyl and 3-oxocyclohexyl.

Two atoms of Ring A may optionally be connected by a bridge. A bridge is a bond, one carbon atom or two carbon atoms which connects two different atoms of Ring A. Particularly the bridge is a bond. Particularly the bridge is one carbon atom. Alternatively the bridge is two carbon atoms. Examples of a “5-7 membered saturated carbocyclic ring wherein 2 atoms of Ring A” are “connected by a bridge” include bicyclo[3.1.0]hexyl, bicyclo[2.2.2]octanyl and bicyclo[2.1.1]hexyl.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

A “nitrogen linked 4-7 membered saturated ring which optionally contains an additional nitrogen, oxygen or sulphur atom” is a saturated monocyclic ring containing 4-7 atoms linked via a nitrogen atom contained in the ring to Ring A. The ring optionally contains an additional heteroatom selected from nitrogen, sulphur or oxygen, wherein a —CH₂— group can optionally be replaced by a —C(O)—, and the optional sulphur atom may be optionally oxidised to form the S-oxides. Particular examples of a “nitrogen linked 4-7 membered saturated ring which optionally contains an additional nitrogen, oxygen or sulphur atom” are piperazin-1-yl, pyrrolidin-1-yl and morpholino.

Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “C₁₋₆alkanoyloxy” include propionyloxy and acetoxy. Examples of “C₁₋₆alkanoylamino” include propionylamino and acetylamino. Examples of “C₂₋₆alkenyl” include vinyl, allyl and 1-propenyl. Examples of “C₂₋₆alkynyl” include ethynyl, 1-propynyl and 2-propynyl. Examples of “N—(C₁₋₆alkyl)sulphamoyl” include N-(methyl)sulphamoyl and N-(ethyl)sulphamoyl. Examples of “N,N—(C₁₋₆alkyl)₂sulphamoyl” include N,N-(dimethyl)sulphamoyl and N-(methyl)-N-(ethyl)sulphamoyl. Examples of “N—(C₁₋₆alkyl)carbamoyl” include methylaminocarbonyl and ethylaminocarbonyl. Examples of “N,N—(C₁₋₆alkyl)₂carbamoyl” include dimethylaminocarbonyl and methylethylaminocarbonyl. Examples of “C₁₋₆alkylsulphonyl” include methylsulphonyl and isopropylsulphonyl. Examples of “C₁₋₆alkylsulphonylamino” include mesylamino and isopropylsulphonylamino. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

An in vivo hydrolysable ester of a compound of the formula (I) containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

Some compounds of the formula (I) may have chiral centres and/or geometric isomeric centres (E- and Z-isomers), and it is to be understood that the invention encompasses all such optical, diastereoisomers and geometric isomers that possess CDK inhibitory activity.

The invention relates to any and all tautomeric forms of the compounds of the formula (I) that possess CDK inhibitory activity.

It is also to be understood that certain compounds of the formula (I) can exist in solvated as well as unsolvated forms such as, for example, hydrated forms. It is to be understood that the invention encompasses all such solvated forms which possess CDK inhibitory activity.

Particular values of variable groups are as follows. Such values may be used where appropriate with any of the definitions, claims or embodiments defined hereinbefore or hereinafter.

Ring A is a 5-7 membered saturated carbocyclic ring.

Ring A is a 5 or 6 membered saturated carbocyclic ring.

Ring A is cyclohexyl.

Ring A is cyclopentyl

Ring A is cyclopentyl or cyclohexyl.

R¹ is selected from amino, sulphamoylamino or a group —R⁶-R⁷; wherein

R⁶ is selected from —N(R¹⁵)—, —N(R¹⁶)C(O)—, —N(R¹⁸)C(O)O— or —N(R²⁵)SO₂—; wherein R¹⁵, R¹⁶, R¹⁸ and R²⁵ are independently hydrogen or C₁₋₆alkyl;

R⁷ is selected from C₁₋₆alkyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;

R²⁷ is selected from C₁₋₆alkyl or heterocyclyl-R³⁰—; wherein if said heterocyclyl contains an —NH— moiety that nitrogen may be optionally substituted by a group selected from R³²;

R²⁸ and R³² are C₁₋₆alkoxycarbonyl; and

R³⁰ is a direct bond.

R¹ is selected from carboxy, amino, sulphamoylamino or a group —R⁶-R⁷; wherein

R⁶ is selected from —N(R¹⁵)—, —C(O)—, —C(O)O—, —N(R¹⁶)C(O)—, —C(O)N(R¹⁷)—, —N(R¹⁸)C(O)O—, —N(R²²)SO₂N(R²³)— or —N(R²⁵)SO₂—; wherein R¹⁵, R¹⁶, R¹⁷, R¹⁸, R²², R²³ and R²⁵ are independently hydrogen or C₁₋₆alkyl;

R⁷ is selected from C₁₋₆alkyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;

R²⁷ is selected from C₁₋₆alkyl, N,N—(C₁₋₆alkyl)₂amino or heterocyclyl-R³⁰—; and wherein if said heterocyclyl contains an —NH— moiety that nitrogen may be optionally substituted by a group selected from R³²;

R²⁸ and R³² are independently selected from C₁₋₆alkyl and C₁₋₆alkoxycarbonyl; and

R³⁰ is a direct bond.

R¹ is selected from amino, sulphamoylamino or a group —R⁶-R⁷; wherein

R⁶ is selected from —N(R¹⁵)—, —N(R¹⁶)C(O)—, —N(R¹⁸)C(O)O— or —N(R²⁵)SO₂—; wherein R¹⁵, R¹⁶, R¹⁸ and R²⁵ are independently hydrogen or methyl;

R⁷ is selected from methyl, ethyl, propyl, t-butyl or piperidinyl; wherein R⁷ may be optionally substituted on carbon by one or more R²⁷; and wherein said piperidinyl may be optionally substituted on nitrogen by a group selected from R²⁸;

R²⁷ is selected from methyl, piperidinyl-R³⁰—, piperazinyl-R³⁰— or morpholino-R³⁰—; wherein said piperidinyl or piperazinyl may be optionally substituted on nitrogen by a group selected from R³²;

R²⁸ and R³² are t-butoxycarbonyl; and

R³⁰ is a direct bond.

R¹ is selected from carboxy, amino, sulphamoylamino or a group —R⁶-R⁷; wherein

R⁶ is selected from —N(R¹⁵)—, —C(O)—, —C(O)O—, —N(R¹⁶)C(O)—, —C(O)N(R¹⁷)—, —N(R¹⁸)C(O)O—, —N(R²²)SO₂N(R²³)— or —N(R²⁵)SO₂—; wherein R¹⁵, R¹⁶, R¹⁷, R¹⁸, R²², R²³ and R²⁵ are independently hydrogen or methyl;

R⁷ is selected from methyl, ethyl, propyl, t-butyl, homopiperazinyl or piperidinyl; wherein R⁷ may be optionally substituted on carbon by one or more R²⁷; and wherein if said heterocyclyl contains an —NH— moiety that nitrogen may be optionally substituted by a group selected from R²⁸;

R²⁷ is selected from methyl, N,N-dimethylamino, pyrrolidinyl-R³⁰—, piperazinyl-R³⁰—, piperidinyl-R³⁰— or morpholino-R³⁰—; and wherein if said heterocyclyl contains an —NH— moiety that nitrogen may be optionally substituted by a group selected from R³²;

R²⁸ and R³² are independently selected from methyl and t-butoxycarbonyl; and

R³⁰ is a direct bond.

R¹ is selected from amino, sulphamoylamino, t-butoxycarbonylamino, mesylamino, dimethylamino, (4-morpholinobutanoyl)amino, 2-(piperidin-4-yl)acetylamino, 2-(N-t-butoxycarbonylpiperidin-4-yl)acetylamino, 3-(piperazin-4-yl)propanoylamino, 3-(1-t-butoxycarbonylpiperazin-4-yl)propanoylamino, 3-(piperidin-4-yl)propanoylamino, 3-(N-t-butoxycarbonylpiperidin-4-yl)propanoylamino, 4-methyl-piperidin-4-ylcarbonylamino N-t-butoxycarbonyl-4-methyl-piperidin-4-ylcarbonylamino, 2-(piperidin-3-yl)acetylamino and 2-(N-t-butoxycarbonylpiperidin-3-yl)acetylamino.

R¹ is selected from amino, carboxy, methoxycarbonyl, sulphamoylamino, N-methylcarbamoyl, N-(2-dimethylaminoethyl)carbamoyl, N-(2-pyrrolidin-1-ylethyl)carbamoyl, N,N-dimethylsulphamoylamino, t-butoxycarbonylamino, mesylamino, dimethylamino, (4-morpholinobutanoyl)amino, 2-(piperidin-4-yl)acetylamino, 2-(N-t-butoxycarbonylpiperidin-4-yl)acetylamino, 3-(piperazin-4-yl)propanoylamino, 3-(1-t-butoxycarbonylpiperazin-4-yl)propanoylamino, 3-(piperidin-4-yl)propanoylamino, 3-(N-t-butoxycarbonylpiperidin-4-yl)propanoylamino, 4-methyl-piperidin-4-ylcarbonylamino, N-t-butoxycarbonyl-4-methyl-piperidin-4-ylcarbonylamino, 2-(pyrrolidin-1-yl)ethylsulphonylamino, 2-(dimethylamino)ethylsulphonylamino, 3-(pyrrolidin-1-yl)propylsulphonylamino, 3-(pyrrolidin-1-yl)propanoylamino, 1-methylhomopiperazin-4-ylcarbonyl, 2-(piperidin-3-yl)acetylamino, 3-(dimethylamino)propanoylamino and 1-methylpiperidin-4-ylcarbonylamino, 2-(N-t-butoxycarbonylpiperidin-3-yl)acetylamino.

q is 0.

R¹, (R²)_{q} and Ring A together form 3-(2-dimethylaminoethylcarbamoyl)cyclopentyl, 3-(2-pyrrolidin-1-ylethylcarbamoyl)cyclopentyl, 3-(4-methyl1,4-diazepane-1-carbonyl)cyclopentyl, 3-(methylcarbamoyl)cyclopentyl, 3-carboxycyclopentyl, 3-(2-dimethylaminoethylsulfonylamino)cyclopentyl, 3-(2-pyrrolidin-1-ylethylsulfonylamino)cyclopentyl, 3-(3-dimethylaminopropanoylamino)cyclopentyl, 3-[(1-methylpiperidine-4-carbonyl)amino]cyclopentyl, 3-aminocyclopentyl, 3-methanesulfonamidocyclopentyl, 3-(2-dimethylaminoethylcarbamoyl)cyclohexyl, 3-(2-pyrrolidin-1-ylethylcarbamoyl)cyclohexyl, 3-(2-pyrrolidin-1-ylethylsulfonylamino)cyclohexyl, 3-(3-dimethylaminopropanoylamino)cyclohexyl, 3-(3-pyrrolidin-1-ylpropanoylamino)cyclohexyl, 3-(3-pyrrolidin-1-ylpropylsulfonylamino)cyclohexyl, 3-(4-methyl1,4-diazepane-1-carbonyl)cyclohexyl, 3-(methylcarbamoyl)cyclohexyl, 3-[(1-methylpiperidine-4-carbonyl)amino]cyclohexyl, 3-aminocyclohexyl, 3-carboxycyclohexyl, 3-methanesulfonamidocyclohexyl, 3-methoxycarbonylcyclohexyl, 4-(2-dimethylaminoethylcarbamoyl)cyclohexyl, 4-(2-pyrrolidin-1-ylethylcarbamoyl)cyclohexyl, 4-(2-pyrrolidin-1-ylethylsulfonylamino)cyclohexyl, 4-(3-dimethylaminopropanoylamino)cyclohexyl, 4-(3-piperazin-1-ylpropanoylamino)cyclohexyl, 4-(3-pyrrolidin-1-ylpropanoylamino)cyclohexyl, 4-(3-pyrrolidin-1-ylpropylsulfonylamino)cyclohexyl, 4-(4-methyl1,4-diazepane-1-carbonyl)cyclohexyl, 4-(4-morpholinobutanoylamino)cyclohexyl, 4-(dimethylsulfamoylamino)cyclohexyl, 4-(methylcarbamoyl)cyclohexyl, 4-(sulfamoylamino)cyclohexyl, 4-(tert-butoxycarbonylamino)cyclohexyl, 4-[(1-methylpiperidine-4-carbonyl)amino]cyclohexyl, 4-[(4-methyl-1-tert-butoxycarbonyl-piperidine-4-carbonyl)amino]cyclohexyl, 4-[(4-methylpiperidine-4-carbonyl)amino]cyclohexyl, 4-[[2-(1-tert-butoxycarbonyl-4-piperidyl)acetyl]amino]cyclohexyl, 4-[[2-(4-piperidyl)acetyl]amino]cyclohexyl, 4-[[2-[1-tert-butoxycarbonyl-3-piperidyl]acetyl]amino]cyclohexyl, 4-[[2-[3-piperidyl]acetyl]amino]cyclohexyl, 4-[[2-[1-tert-butoxycarbonyl-3-piperidyl]acetyl]amino]cyclohexyl, 4-[[2-[3-piperidyl]acetyl]amino]cyclohexyl, 4-[3-(1-tert-butoxycarbonyl-4-piperidyl)propanoylamino]cyclohexyl, 4-[3-(4-piperidyl)propanoylamino]cyclohexyl, 4-[3-(4-tert-butoxycarbonylpiperazin-1-yl)propanoylamino]cyclohexyl, 4-aminocyclohexyl, 4-carboxycyclohexyl, 4-dimethylaminocyclohexyl and 4-methanesulfonamidocyclohexyl.

R³ is halo.

R³ is fluoro.

R³ is chloro.

R³ is bromo.

R³ is cyano.

R³ is amino.

n is 0.

n is 1.

n is 0 or 1.

R⁴ is isopropyl.

R⁵ is methyl.

Therefore in a further aspect of the invention there is provided a compound of formula (I) (as depicted above) wherein:

Ring A is a 5-7membered saturated carbocyclic ring;

R¹ is selected from amino, sulphamoylamino or a group —R⁶-R⁷;

q is 0;

n is 0;

R⁴ is isopropyl;

R⁵ is methyl;

R⁶ is selected from —N(R¹⁵)—, —N(R¹⁶)C(O)—, —N(R¹⁸)C(O)O— or —N(R²⁵)SO₂—; wherein R¹⁵, R¹⁶, R¹⁸ and R²⁵ are independently hydrogen or C₁₋₆alkyl;

R⁷ is selected from C₁₋₆alkyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;

R²⁷ is selected from C₁₋₆alkyl or heterocyclyl-R³⁰—; wherein if said heterocyclyl contains an —NH— moiety that nitrogen may be optionally substituted by a group selected from R³²;

R²⁸ and R³² are C₁₋₆alkoxycarbonyl; and

R³⁰ is a direct bond;

or a pharmaceutically acceptable salt or an in vivo hydrolysable ester thereof.

Therefore in a further aspect of the invention there is provided a compound of formula (I) (as depicted above) wherein:

Ring A is a 5 or 6 membered saturated carbocyclic ring;

R¹ is selected from carboxy, amino, sulphamoylamino or a group —R⁶-R⁷;

q is 0;

R³ is halo;

n is 0 or 1;

R⁴ is isopropyl;

R⁵ is methyl;

R⁶ is selected from —N(R¹⁵)—, —C(O)—, —C(O)O—, —N(R¹⁶)C(O)—, —C(O)N(R¹⁷)—, —N(R¹⁸)C(O)O—, —N(R²²)SO₂N(R²³)— or —N(R²⁵)SO₂—; wherein R¹⁵, R¹⁶, R¹⁷, R¹⁸, R²², R²³ and R²⁵ are independently hydrogen or C₁₋₆alkyl;

R⁷ is selected from C₁₋₆alkyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;

R²⁷ is selected from C₁₋₆alkyl, N,N—(C₁₋₆alkyl)₂amino or heterocyclyl-R³⁰—; and wherein if said heterocyclyl contains an —NH— moiety that nitrogen may be optionally substituted by a group selected from R³²;

R²⁸ and R³² are independently selected from C₁₋₆alkyl and C₁₋₆alkoxycarbonyl; and

R³⁰ is a direct bond;

or a pharmaceutically acceptable salt or an in vivo hydrolysable ester thereof.

Therefore in a further aspect of the invention there is provided a compound of formula (I) (as depicted above) wherein:

Ring A is cyclopentyl or cyclohexyl;

R¹ is selected from amino, carboxy, methoxycarbonyl, sulphamoylamino, N-methylcarbamoyl, N-(2-dimethylaminoethyl)carbamoyl, N-(2-pyrrolidin-1-ylethyl)carbamoyl, N,N-dimethylsulphamoylamino, t-butoxycarbonylamino, mesylamino, dimethylamino, (4-morpholinobutanoyl)amino, 2-(piperidin-4-yl)acetylamino, 2-(N-t-butoxycarbonylpiperidin-4-yl)acetylamino, 3-(piperazin-4-yl)propanoylamino, 3-(1-t-butoxycarbonylpiperazin-4-yl)propanoylamino, 3-(piperidin-4-yl)propanoylamino, 3-(N-t-butoxycarbonylpiperidin-4-yl)propanoylamino, 4-methyl-piperidin-4-ylcarbonylamino, N-t-butoxycarbonyl-4-methyl-piperidin-4-ylcarbonylamino, 2-(pyrrolidin-1-yl)ethylsulphonylamino, 2-(dimethylamino)ethylsulphonylamino, 3-(pyrrolidin-1-yl)propylsulphonylamino, 3-(pyrrolidin-1-yl)propanoylamino, 1-methylhomopiperazin-4-ylcarbonyl, 2-(piperidin-3-yl)acetylamino, 3-(dimethylamino)propanoylamino and 1-methylpiperidin-4-ylcarbonylamino, 2-(N-t-butoxycarbonylpiperidin-3-yl)acetylamino;

q is 0;

R³ is halo;

n is 0 or 1;

R⁴ is isopropyl;

R⁵ is methyl;

or a pharmaceutically acceptable salt or an in vivo hydrolysable ester thereof.

In another aspect of the invention, preferred compounds of the invention are any one of the Examples or a pharmaceutically acceptable salt or an in vivo hydrolysable ester thereof.

In another aspect of the invention, particular compounds of the invention are any one of Examples 2, 3, 6, 45, 46, 47, 54, 56, 57 and 58 or a pharmaceutically acceptable salt or an in vivo hydrolysable ester thereof.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt or an in vivo hydrolysable ester thereof which process (wherein variable groups are, unless otherwise specified, as defined in formula (I)) comprises of:

Process a) reaction of a pyrimidine of formula (II):

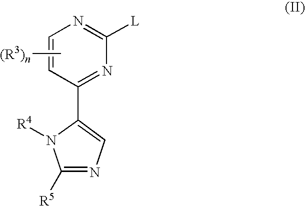

wherein L is a displaceable group; with an amine of formula (III):

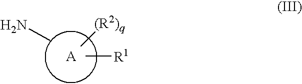

or
Process b) reacting a compound of formula (IV):

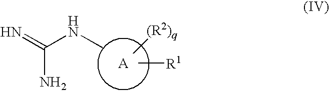

with a compound of formula (V):

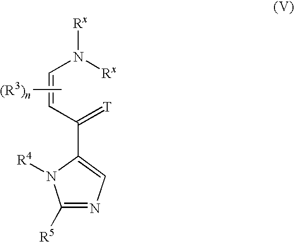

wherein T is O or S; R^{x} may be the same or different and is selected from C₁₋₆alkyl; or
Process c) reacting a pyrimidine of formula (VI):

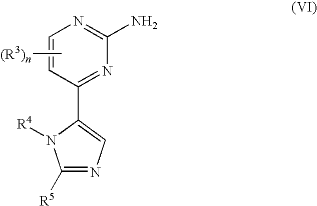

with a compound of formula (VII):

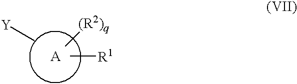

where Y is a displaceable group;
and thereafter if necessary:
i) converting a compound of the formula (I) into another compound of the formula (I);
ii) removing any protecting groups;
iii) forming a pharmaceutically acceptable salt or in vivo hydrolysable ester.

L is a displaceable group, suitable values for L are for example, a halogen or sulphonyloxy group, for example a chloro, bromo, methanesulphonyloxy or toluene-4-sulphonyloxy group.

Y is a displaceable group, suitable values for Y are for example, a halogeno or sulphonyloxy group, for example a bromo, iodo or trifluoromethanesulphonyloxy group. Preferably Y is iodo.

Specific reaction conditions for the above reactions are as follows.

Process a) Pyrimidines of formula (II) and amines of formula (III) may be reacted together in a suitable solvent such as tetrahydrofuran, N-methylpyrrolidinone or isopropyl alcohol, or can be reacted together neat, at a temperature in the range of 25-200° C., particularly in the range of 60-160° C. The reaction may be conducted in the presence of a suitable base such as, for example, N,N-diisopropylethylamine, sodium hydride or potassium carbonate.

Pyrimidines of the formula (II) where L is chloro may be prepared according to Scheme 1:

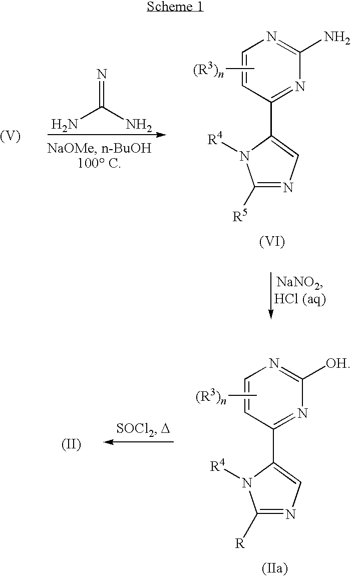

Amines of formula (III) are commercially available compounds, or they are known in the literature, or they are prepared by standard processes known in the art.

Process b) Compounds of formula (IV) and compounds of formula (V) are reacted together in a suitable solvent such as N-methylpyrrolidinone or butanol at a temperature in the range of 100-200° C., preferably in the range of 150-170° C. The reaction is preferably conducted in the presence of a suitable base such as, for example, sodium hydride, sodium methoxide or potassium carbonate.

Compounds of formula (V) may be prepared according to Scheme 2:

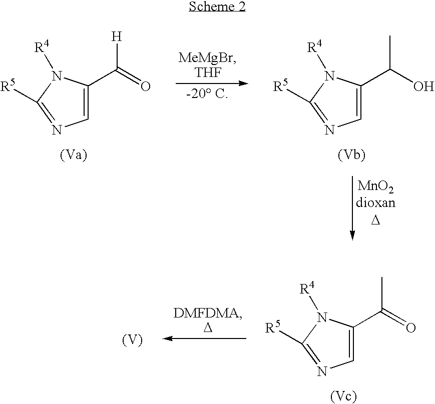

Compounds of formula (IV) and (Va) are commercially available compounds, or they are known in the literature, or they are prepared by standard processes known in the art.

Process c) Compounds of formula (VI) and amines of formula (VII) may be reacted together under the conditions described in Process a.

The synthesis of compounds of formula (VI) is described in Scheme 1.

Compounds of formula (VII) are commercially available compounds, or they are known in the literature, or they are prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cell-proliferation activity such as anti-cancer activity which is believed to arise from the CDK inhibitory activity of the compound. These properties may be assessed, for example, using the procedure set out below:—

### Assay

The following abbreviations have been used:—

HEPES is N-[2-Hydroxyethyl]piperazine-N′-[2-ethanesulfonic acid]

### DTT is Dithiothreitol

PMSF is Phenylmethylsulphonyl fluoride

The compounds were tested in an in vitro kinase assay in 96 well format using Scintillation Proximity Assay (SPA—obtained from Amersham) for measuring incorporation of [γ-33-P]-Adenosine Triphosphate into a test substrate (GST-Retinoblastoma protein; GST-Rb). In each well was placed the compound to be tested (diluted in DMSO and water to correct concentrations) and in control wells either roscovitine as an inhibitor control or DMSO as a positive control.

Approximately 0.2 μl of CDK2/Cyclin E partially-purified enzyme (amount dependent on enzyme activity) diluted in 25 μl incubation buffer was added to each well then 20 μl of GST-Rb/ATP/ATP33 mixture (containing 0.5 μg GST-Rb and 0.2 μM ATP and 0.14 μCi [γ-33-P]-Adenosine Triphosphate in incubation buffer), and the resulting mixture shaken gently, then incubated at room temperature for 60 mins.

To each well was then added 150 μL stop solution containing (0.8 mg/well of Protein A-PVT SPA bead (Amersham)), 20 pM/well of Anti-Glutathione Transferase, Rabbit IgG (obtained from Molecular Probes), 61 mM EDTA and 50 mM HEPES pH 7.5 containing 0.05% sodium azide.

The plates were sealed with Topseal-S plate sealers, left for two hrs then spun at 2500 rpm, 1124×g., for 5 mins. The plates were read on a Topcount for 30 seconds per well.

The incubation buffer used to dilute the enzyme and substrate mixes contained 50 mM HEPES pH7.5, 10 mM MnCl₂, 1 mM DTT, 100 μM Sodium vanadate, 100 μM NaF, 10 mM Sodium Glycerophosphate, BSA (1 mg/ml final).

### Test Substrate

In this assay only part of the retinoblastoma protein (Science 1987 Mar. 13; 235(4794):1394-1399; Lee W. H., Bookstein R., Hong F., Young L. J., Shew J. Y., Lee E. Y.) was used, fused to a GST tag. PCR of retinoblastoma gene encoding amino acids 379-928 (obtained from retinoblastoma plasmid ATCC pLRbRNL) was performed, and the sequence cloned into pGEx 2T fusion vector (Smith D. B. and Johnson, K. S. Gene 67, 31 (1988); which contained a tac promoter for inducible expression, internal lac I^{q} gene for use in any E. Coli host, and a coding region for thrombin cleavage—obtained from Pharmacia Biotech) which was used to amplify amino acids 792-928. This sequence was again cloned into pGEx 2T.

The retinoblastoma 792-928 sequence so obtained was expressed in E. Coli (BL21 (DE3) pLysS cells) using standard inducible expression techniques, and purified as follows.

E. coli paste was resuspended in 10 ml/g of NETN buffer (50 mM Tris pH 7.5, 120 mM NaCl, 1 mM EDTA, 0.5% v/v NP-40, 1 mM PMSF, 1 ug/ml leupeptin, 1 ug/ml aprotinin and 1 ug/ml pepstatin) and sonicated for 2×45 seconds per 100 ml homogenate. After centrifugation, the supernatant was loaded onto a 10 ml glutathione Sepharose column (Pharmacia Biotech, Herts, UK), and washed with NETN buffer. After washing with kinase buffer (50 mM HEPES pH 7.5, 10 mM MgCl2, 1 mM DTT, 1 mM PMSF, 1 ug/ml leupeptin, 1 ug/ml aprotinin and 1 ug/ml pepstatin) the protein was eluted with 50 mM reduced glutathione in kinase buffer. Fractions containing GST-Rb (792-927) were pooled and dialysed overnight against kinase buffer. The final product was analysed by Sodium Dodeca Sulfate (SDS) PAGE (Polyacrylamide gel) using 8-16% Tris-Glycine gels (Novex, San Diego, USA).

### CDK2 and Cyclin E

The open reading frames of CDK2 and Cyclin E were isolated by reverse transcriptase-PCR using HeLa cell and activated T cell mRNA as a template and cloned into the insect expression vector pVL1393 (obtained from Invitrogen 1995 catalogue number: V1392-20). CDK2 and cyclin E were then dually expressed [using a standard virus Baculogold co-infection technique] in the insect SF21 cell system (Spodoptera Frugiperda cells derived from ovarian tissue of the Fall Army Worm—commercially available).

### Example Production of Cyclin E/CDK2

The following Example provides details of the production of Cyclin E/CDK2 in SF21 cells (in TC100+10% FBS(TCS)+0.2% Pluronic) having dual infection MOI 3 for each virus of Cyclin E & CDK2.

SF21 cells grown in a roller bottle culture to 2.33×10⁶ cells/ml were used to inoculate 10×500 ml roller bottles at 0.2×10E6 cells/ml. The roller bottles were incubated on a roller rig at 28° C.

After 3 days (72 hrs.) the cells were counted, and the average from 2 bottles found to be 1.86×10E6 cells/ml. (99% viable). The cultures were then infected with the dual viruses at an MOI 3 for each virus.

The viruses were mixed together before addition to the cultures, and the cultures returned to the roller rig 28° C.

After 2 days (48 hrs.) post infection the 5 Litres of culture was harvested. The total cell count at harvest was 1.58×10E6 cells/ml. (99% viable). The cells were spun out at 2500 rpm, 30 mins., 4° C. in Heraeus Omnifuge 2.0 RS in 250 ml. lots. The supernatant was discarded.

### Partial Co-Purification of CDK2 and Cyclin E

Sf21 cells were resuspended in lysis buffer (50 mM Tris pH 8.2, 10 mM MgCl₂, 1 mM DTT, 10 mM glycerophosphate, 0.1 mM sodium orthovanadate, 0.1 mM NaF, 1 mM PMSF, 1 ug/ml leupeptin and 1 ug/ml aprotinin) and homogenised for 2 mins in a 10 ml Dounce homgeniser. After centrifugation, the supernatant was loaded onto a Poros HQ/M 1.4/100 anion exchange column (PE Biosystems, Hertford, UK). CDK2 and Cyclin E were coeluted at the beginning of a 0-1M NaCl gradient (run in lysis buffer minus protease inhibitors) over 20 column volumes. Co-elution was checked by western blot using both anti-CDK2 and anti-Cyclin E antibodies (Santa Cruz Biotechnology, California, US).

By analogy, assays designed to assess inhibition of CDK1 and CDK4 may be constructed. CDK2 (EMBL Accession No. X62071) may be used together with Cyclin A or Cyclin E (see EMBL Accession No. M73812), and further details for such assays are contained in PCT International Publication No. WO99/21845, the relevant Biochemical & Biological Evaluation sections of which are hereby incorporated by reference.

Although the pharmacological properties of the compounds of the formula (I) vary with structural change, in general activity possessed by compounds of the formula (I) may be demonstrated at IC₅₀ concentrations or doses in the range 250 μM to 1 nM.

When tested in the above in-vitro assay the CDK2 inhibitory activity of Example 4 was measured as IC₅₀=159 nM.

According to a further aspect of the invention there is provided a pharmaceutical composition which comprises a pyrimidine derivative of the formula (I), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt or in vivo hydrolysable ester thereof, as defined hereinbefore for use in a method of treatment of the human or animal body by therapy.

We have found that the compounds defined in the present invention, or a pharmaceutically acceptable salt or in vivo hydrolysable ester thereof, are effective cell cycle inhibitors (anti-cell proliferation agents), which property is believed to arise from their CDK inhibitory properties. Accordingly the compounds of the present invention are expected to be useful in the treatment of diseases or medical conditions mediated alone or in part by CDK enzymes, i.e. the compounds may be used to produce a CDK inhibitory effect in a warm-blooded animal in need of such treatment. Thus the compounds of the present invention provide a method for treating the proliferation of malignant cells characterised by inhibition of CDK enzymes, i.e. the compounds may be used to produce an anti-proliferative and potentially apoptotic effect mediated alone or in part by the inhibition of CDKs. Particularly, an inhibitory effect is produced by preventing entry into or progression through the S phase by inhibition of CDK2, CDK4 and/or CDK6, especially CDK2 and entry into or progression through M phase by inhibition of CDK1. Apoptotic effects may also be envisaged through down-regulation of RNA polymerase II activity by inhibition of CDK1, CDK7, CDK8 and in particular, CDK9. Such a compound of the invention is expected to possess a wide range of anti-cancer properties as CDKs have been implicated in many common human cancers such as leukaemia and breast, lung, colon, rectal, stomach, prostate, bladder, pancreas and ovarian cancer. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, prostate and pancreas. In particular such compounds of the invention are expected to slow advantageously the growth of primary and recurrent solid tumours of, for example, the colon, breast, prostate, lungs and skin. More particularly such compounds of the invention, or a pharmaceutically acceptable salt or in vivo hydrolysable ester thereof, are expected to inhibit the growth of those primary and recurrent solid tumours which are associated with CDKs, especially those tumours which are significantly dependent on CDKs for their growth and spread, including for example, certain tumours of the colon, breast, prostate, lung, vulva and skin.

It is further expected that a compound of the present invention will possess activity against other cell-proliferation diseases in a wide range of other disease states including leukaemias, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Thus according to this aspect of the invention there is provided a compound of the formula (I), or a pharmaceutically acceptable salt or in vivo hydrolysable ester thereof, as defined hereinbefore for use as a medicament.

In a further aspect of the invention there is provided the use of a compound of the formula (I), or a pharmaceutically acceptable salt or in vivo hydrolysable ester thereof, as defined hereinbefore in the manufacture of a medicament for the production of a cell cycle inhibitory effect.

In one aspect of the invention, where a cell cycle inhibitory effect is referred to this refers to inhibition of CDK1. In a further aspect of the invention, this refers to inhibition of CDK2. In a further aspect of the invention, this refers to inhibition of CDK4. In a further aspect of the invention, this refers to inhibition of CDK5. In a further aspect of the invention, this refers to inhibition of CDK6. In a further aspect of the invention, this refers to inhibition of CDK7. In a further aspect of the invention, this refers to inhibition of CDK8. In a further aspect of the invention, this refers to inhibition of CDK9.

In a further aspect of the invention there is provided the use of a compound of the formula (I), or a pharmaceutically acceptable salt or in vivo hydrolysable ester thereof, as defined hereinbefore in the manufacture of a medicament for the production of an anti-cell-proliferation effect.

In a further aspect of the invention there is provided the use of a compound of the formula (I), or a pharmaceutically acceptable salt or in vivo hydrolysable ester thereof, as defined hereinbefore in the manufacture of a medicament for the production of a CDK2 inhibitory effect.

In a further aspect of the invention there is provided the use of a compound of the formula (I), or a pharmaceutically acceptable salt or in vivo hydrolysable ester thereof, as defined hereinbefore in the manufacture of a medicament for the treatment of cancer.

In a further aspect of the invention there is provided the use of a compound of the formula (I), or a pharmaceutically acceptable salt or in vivo hydrolysable ester thereof, as defined hereinbefore in the manufacture of a medicament for the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the manufacture of a medicament for the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided a method of producing a cell cycle inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing a CDK2 inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use as a medicament.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a cell cycle inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of an anti-cell-proliferation effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a CDK2 inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of leukaemia or lymphoid malignancies or cancer of the breast, lung, colon, rectum, stomach, liver, kidney, prostate, bladder, pancreas, vulva, skin or ovary.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided the use of a compound of the formula (I), or a pharmaceutically acceptable salt or in vivo hydrolysable ester thereof, as defined hereinbefore, in the production of a cell cycle inhibitory effect.

In a further aspect of the invention there is provided the use of a compound of the formula (I), or a pharmaceutically acceptable salt or in vivo hydrolysable ester thereof, as defined hereinbefore, in the production of an anti-cell-proliferation effect.

In a further aspect of the invention there is provided the use of a compound of the formula (I), or a pharmaceutically acceptable salt or in vivo hydrolysable ester thereof, as defined hereinbefore, in the production of a CDK2 inhibitory effect.

In a further aspect of the invention there is provided the use of a compound of the formula (I), or a pharmaceutically acceptable salt or in vivo hydrolysable ester thereof, as defined hereinbefore, in the treatment of cancer.

In a further aspect of the invention there is provided the use of a compound of the formula (I), or a pharmaceutically acceptable salt or in vivo hydrolysable ester thereof, as defined hereinbefore in the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Preventing cells from entering DNA synthesis by inhibition of essential S-phase initiating activities such as CDK2 initiation may also be useful in protecting normal cells of the body from toxicity of cycle-specific pharmaceutical agents. Inhibition of CDK2 or 4 will prevent progression into the cell cycle in normal cells which could limit the toxicity of cycle-specific pharmaceutical agents which act in S-phase, G2 or mitosis. Such protection may result in the prevention of hair loss normally associated with these agents.

Therefore in a further aspect of the invention there is provided a compound of formula (I) as defined above or a pharmaceutically acceptable salt or in vivo hydrolysable ester thereof for use as a cell protective agent.

Therefore in a further aspect of the invention there is provided a compound of formula (I) as defined above or a pharmaceutically acceptable salt or in vivo hydrolysable ester thereof for use in preventing hair loss arising from the treatment of malignant conditions with pharmaceutical agents.

Examples of pharmaceutical agents for treating malignant conditions that are known to cause hair loss include alkylating agents such as ifosfamide and cyclophosphamide; antimetabolites such as methotrexate, 5-fluorouracil, gemcitabine and cytarabine; vinca alkaloids and analogues such as vincristine, vinbalstine, vindesine, vinorelbine; taxanes such as paclitaxel and docetaxel; topoisomerase I inhibitors such as irintotecan and topotecan; cytotoxic antibiotics such as doxorubicin, daunorubicin, mitoxantrone, actinomycin-D and mitomycin; and others such as etoposide and tretinoin.

In another aspect of the invention, the compound of formula (I), or a pharmaceutically acceptable salt or in vivo hydrolysable ester thereof, may be administered in association with a one or more of the above pharmaceutical agents. In this instance the compound of formula (I) may be administered by systemic or non systemic means. Particularly the compound of formula (I) my may administered by non-systemic means, for example topical administration.

Therefore in an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof.

In an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof in simultaneous, sequential or separate administration with an effective amount of said pharmaceutical agent.

According to a further aspect of the invention there is provided a pharmaceutical composition for use in preventing hair loss arising from the treatment of malignant conditions with pharmaceutical agents which comprises a compound of formula (I), or a pharmaceutically acceptable salt or in vivo hydrolysable ester thereof, and said pharmaceutical agent, in association with a pharmaceutically acceptable diluent or carrier.

According to a further aspect of the present invention there is provided a kit comprising a compound of formula (I), or a pharmaceutically acceptable salt or in vivo hydrolysable ester thereof, and a pharmaceutical agent for treating malignant conditions that is known to cause hair loss.

According to a further aspect of the present invention there is provided a kit comprising:

a) a compound of formula (I), or a pharmaceutically acceptable salt or in vivo hydrolysable ester thereof, in a first unit dosage form;
b) a pharmaceutical agent for treating malignant conditions that is known to cause hair loss; in a second unit dosage form; and
c) container means for containing said first and second dosage forms.

According to another feature of the invention there is provided the use of a compound of the formula (I), or a pharmaceutically acceptable salt or in vivo hydrolysable ester thereof, in the manufacture of a medicament for the prevention of hair loss during treatment of malignant conditions with pharmaceutical agents.

According to a further aspect of the present invention there is provided a combination treatment for the prevention of hair loss comprising the administration of an effective amount of a compound of the formula (I), or a pharmaceutically acceptable salt or in vivo hydrolysable ester thereof, optionally together with a pharmaceutically acceptable diluent or carrier, with the simultaneous, sequential or separate administration of an effective amount of a pharmaceutical agent for treatment of malignant conditions to a warm-blooded animal, such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The CDK inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may cover three main categories of therapeutic agent:

(i) other cell cycle inhibitory agents that work by the same or different mechanisms from those defined hereinbefore;
(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors); and
(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan). According to this aspect of the invention there is provided a pharmaceutical product comprising a compound of the formula (I) as defined hereinbefore and an additional anti-tumour substance as defined hereinbefore for the conjoint treatment of cancer.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-d₆) as solvent unless otherwise indicated;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported;
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xvi) the following abbreviations have been used:
BOC tert-butoxy carbonyl;
IPA isopropyl alcohol;
THF tetrahydrofuran;
DIPEA N,N-diisopropylethylamine;
DMF N,N-dimethylformamide;
EtOAc ethyl acetate;
MeOH methanol;
ether diethyl ether;
HATU O-(7-azabenzotriazol-1-yl)-1,1-3,3-tetramethyluronium hexafluorophosphate;
DCM dichloromethane;
DMSO dimethylsulphoxide; and
TFA trifluoroacetic acid;
   (xvii) PTFE filters used for filtration are manufactured by Gelman® and consist of a 0.45 μM membrane filter cup. These are available from Fisher-Scientific UK (Part Code 09730155); and
   (xviii) where an SCX-2 column is referred to, this means an “ion exchange” extraction cartridge for adsorption of basic compounds, i.e. a polypropylene tube containing a benzenesulphonic acid based strong cation exchange sorbent, used according to the manufacturers instructions obtained from International Sorbent Technologies Limited, Dyffryn Business Park, Hengeod, Mid Glamorgan, UK, CF82 7RJ.

### Example 1

### tert-Butyl trans-N-[4-[[4-(2-methyl-3-propan-2-yl-imidazol-4-yl)pyrimidin-2-yl]amino]cyclohexyl]carbamate

2-Chloro-4-(2-methyl-3-propan-2-yl-imidazol-4-yl)pyrimidine (Method 2, 1.9 g, 8 mmol), tert-butyl trans-N-(4-aminocyclohexyl)carbamate (2.58 g, 12 mmol), triethylamine (3.35 ml, 24 mmol) and IPA (20 ml) were combined and heated at reflux (85° C.) for 5 days. On cooling to ambient temperature the mixture set solid. The reaction mixture was diluted with IPA and the resulting precipitate filtered and washed with IPA. The filtrate was evaporated and triturated with IPA and a second crop of material collected by filtration. The two precipitates were combined, dissolved in DCM/MeOH, insoluble material filtered off, and the solution passed through a short pad of silica, eluting with a gradient of 0-10% MeOH/DCM. Fractions containing product were combined and evaporated to give the title compound as a white solid (1.68 g, 50%). NMR (400.132 MHz, CDCl₃) 1.22 (m, 4H), 1.38 (s, 9H), 1.48 (d, 6H), 2.05 (m, 4H), 2.50 (s, 3H), 3.40 (m, 1H), 3.69 (m, 1H), 4.32 (m, 1H), 4.83 (m, 1H), 5.54 (m, 1H), 6.64 (d, 1H), 7.23 (s, 1H), 8.12 (d, 1H); MH+ 415.

### Example 2

### N-[trans-4-(2-Methyl-3-propan-2-yl-imidazol-4-yl)pyrimidin-2-yl]cyclohexane-1,4-diamine

tert-Butyl trans-N-[4-[[4-(2-methyl-3-propan-2-yl-imidazol-4-yl)pyrimidin-2-yl]amino]cyclohexyl]carbamate (Example 1, 1.68 g, 4 mmol) was suspended in dioxane (20 ml), 4M HCl in dioxane (20 ml) was added and the reaction mixture stirred for 3 days at ambient temperature. The resulting precipitate was filtered off and dissolved in water, neutralised to pH 9 with 2M aq. NaOH and extracted with DCM (2×75 ml). The organic phase was gravity filtered through a PTFE cup and the solvent evaporated. The aqueous phase was acidified with 2M aqueous HCl to pH 4 then added to a 50 g SCX-2 column pre-treated with MeOH. The column was flushed with water (2 column volumes), MeOH (1 column volume) and the product eluted with 2M ammonia in MeOH. The eluent was combined with the previous organic phase and solvents evaporated to give a clear gum. Ether was added to the residue and solvent was re-evaporated and the resultant material subjected to a high vacuum to give the title compound as a white foam. (1.26 g, 99%). ¹H NMR (400.132 MHz, CDCl₃) 1.17 (m, 6H), 1.49 (d, 6H), 1.85 (m, 2H), 2.07 (m, 2H), 2.50 (s, 3H), 2.68 (m, 1H), 3.69 (m, 1H), 4.82 (m, 1H), 5.58 (m, 1H), 6.64 (d, 1H), 7.24 (s, 1H), 8.11 (d, 1H). MH+ 315.

### Example 3

### N-[trans-4-[[4-(2-Methyl-3-propan-2-yl-imidazol-4-yl)pyrimidin-2-yl]amino]cyclohexyl]methanesulfonamide

To a solution of N-[trans-4-(2-methyl-3-propan-2-yl-imidazol-4-yl)pyrimidin-2-yl]cyclohexane-1,4-diamine (Example 2, 60 mg, 0.2 mmol) in DCM (2 ml), was added triethylamine (0.08 ml, 0.57 mmol) followed by a solution of methane sulfonyl chloride (0.019 ml, 0.25 mmol) in DCM (1 ml). The resulting solution was stirred at ambient temperature for 2 hrs, washed with water and gravity filtered through a PTFE cup. The solvent was evaporated and the residue was dissolved in DCM and purified on silica, eluting with a shallow gradient of 0-5% MeOH/DCM then 5% MeOH/DCM. Fractions containing the product were combined and evaporated to give the title compound as a white solid (20 mg, 26%). NMR (400.132 MHz, CDCl₃) 1.31 (m, 4H), 1.49 (d, 6H), 2.11 (m, 4H), 2.50 (s, 3H), 2.92 (s, 3H), 3.29 (m, 1H), 3.71 (m, 1H), 4.07 (d, 1H), 4.82 (m, 1H), 5.50 (m, 1H), 6.66 (d, 1H), 7.24 (s, 1H), 8.12 (d, 1H); MH+ 393.

### Example 4

### trans-N′,N′-Dimethyl-N-[4-(2-methyl-3-propan-2-yl-imidazol-4-yl)pyrimidin-2-yl]cyclohexane-1,4-diamine

To a solution of N-[trans-4-(2-methyl-3-propan-2-yl-imidazol-4-yl)pyrimidin-2-yl]cyclohexane-1,4-diamine (Example 2, 60 mg, 0.2 mmol) in THF (2 ml), was added acetic acid (0.011 ml, 0.2 mmol) followed by 37% aqueous formaldehyde solution (1 ml). The reaction mixture was stirred at ambient temperature for 30 mins. Sodium triacetoxyborohydride (121 mg, 0.6 mmol) was added and the reaction was stirred for 2 hrs. The solvents were evaporated and the residue was neutralised with saturated aqueous sodium bicarbonate, extracted with DCM, gravity filtered through a PTFE cup and the organic extract evaporated. The resultant material was dissolved in DCM and purified on silica eluting with 10% 2M ammonia in MeOH/DCM. Fractions containing product were combined and evaporated to a clear gum. Ether was added and re-evaporated to give the title compound as a glassy white solid. (28 mg, 41%). NMR (400.132 MHz, CDCl₃) 1.23 (m, 4H), 1.49 (d, 6H), 1.93 (m, 2H), 2.13 (m, 3H), 2.24 (s, 6H), 2.50 (s, 3H), 3.67 (m, 1H), 4.83 (m, 1H), 5.58 (m, 1H), 6.64 (d, 1H), 7.24 (s, 1H), 8.11 (d, 1H); MH+ 343.

### Example 5

### N-[trans-4-[[4-(2-Methyl-3-propan-2-yl-imidazol-4-yl)pyrimidin-2-yl]amino]cyclohexyl]-4-morpholin-4-yl-butanamide

N-[trans-4-(2-Methyl-3-propan-2-yl-imidazol-4-yl)pyrimidin-2-yl]cyclohexane-1,4-diamine (Example 2, 60 mg, 0.2 mmol), 4-morpholin-4-ylbutanoic acid hydrochloride (Method 3, 38 mg, 0.23 mmol), HATU (87 mg, 0.23 mmol), DIPEA (0.13 ml, 0.76 mmol) and DMF (2 ml) were combined and stirred at ambient temperature overnight. The solvents were evaporated and the residue partitioned between DCM (2 ml) and saturated aqueous sodium bicarbonate (2 ml), gravity filtered through a PTFE cup and DCM evaporated. The resultant material was dissolved in DCM and purified on silica, eluting on a gradient of 0-5% 2M ammonia in MeOH. Fractions containing product were combined and evaporated to give the title compound as a white solid. (61 mg, 68%). NMR (400.132 MHz, CDCl₃) 1.25 (m, 4H), 1.49 (d, 6H), 1.77 (m, 2H), 1.97 (m, 3H), 2.10 (m, 2H), 2.19 (m, 2H), 2.37 (m, 2H), 2.43 (m, 2H), 2.50 (s, 3H), 2.96 (m, 2H), 3.71 (m, 5H), 4.83 (m, 1H), 5.52 (m, 1H), 5.73 (m, 1H), 6.65 (d, 1H), 7.23 (s, 1H), 8.12 (d, 1H); MH+ 470.

### Example 6

### 4-(2-Methyl-3-propan-2-yl-imidazol-4-yl)-2-[[trans-4-(sulfamoylamino)cyclohexyl]amino]pyrimidine

N-[trans-4-(2-Methyl-3-propan-2-yl-imidazol-4-yl)pyrimidin-2-yl]cyclohexane-1,4-diamine (Example 2, 70 mg, 0.22 mmol), sulfamide (214 mg, 2.23 mmol) and dioxane (2 ml) were combined and heated at reflux for 16 hrs. The reaction was allowed to cool to ambient temperature and diluted with water (50 ml), saturated aqueous sodium bicarbonate solution (50 ml) and DCM (100 ml). Insoluble material filtered and washed with water, the organic phase was washed with further water then evaporated and combined with the filtered insoluble material. Combined materials were triturated with ether and the resulting solid filtered to give the title compound as a beige solid (64 mg, 73%). NMR (400.132 MHz) 1.31 (m, 4H), 1.50 (d, 6H), 1.97 (m, 4H), 2.50 (s, 3H), 3.05 (m, 1H), 3.62 (m, 1H), 5.72 (m, 1H), 6.45 (m, 3H), 6.78 (d, 1H), 7.00 (m, 1H), 7.35 (s, 1H), 8.18 (d, 1H); MH+ 394.

### Example 7

### 2-[[trans-4-(Dimethylsulfamoylamino)cyclohexyl]amino]-4-(2-methyl-3-propan-2-yl-imidazol-4-yl)pyrimidine

Triethylamine (0.08 ml, 0.6 mmol) was added to a solution of N-[trans-4-(2-methyl-3-propan-2-yl-imidazol-4-yl)pyrimidin-2-yl]cyclohexane-1,4-diamine (Example 2; 63 mg, 0.2 mmol) dissolved in DCM (1 ml). A solution of dimethylsulfamoyl chloride (0.03 ml, 0.24 mmol) in DCM (1 ml) was added dropwise and the solution stirred at ambient temperature for 16 hrs. Additional dimethylsulfamoyl chloride (0.03 ml, 0.24 mmol) was added and the reaction stirred for a further 3 hrs. Water (2 ml) was then added, the reaction mixture shaken and gravity filtered through a PTFE cup. The resulting solution was purified on silica, eluting with a gradient of 0-5% MeOH/DCM, to give the title compound as a colourless solid (19 mg, 23%). NMR (400.132 MHz, CDCl₃) 1.27 (m, 4H), 1.48 (d, 6H), 2.10 (m, 4H), 2.50 (s, 3H), 2.73 (s, 6H), 3.17 (m, 1H), 3.69 (m, 1H), 4.15 (m, 1H), 4.92 (m, 1H), 5.51 (m, 1H), 6.65 (d, 1H), 7.23 (s, 1H), 8.11 (d, 1H); MH+ 422.

### Example 8

### tert-Butyl 4-[[trans-4-[[4-(2-methyl-3-propan-2-yl-imidazol-4-yl)pyrimidin-2-yl]amino]cyclohexyl]carbamoylmethyl]piperidine-1-carboxylate

N-[trans-4-(2-Methyl-3-propan-2-yl-imidazol-4-yl)pyrimidin-2-yl]cyclohexane-1,4-diamine (Example 2, 100 mg, 0.32 mmol), 1-(1,1-dimethylethoxy)carbonyl)-4-piperidineacetic acid (93 mg, 0.38 mmol), HATU (145 mg, 0.38 mmol), DIPEA (0.22 ml, 1.27 mmol) and DMF (4 ml) were combined and stirred overnight at ambient temperature. Solvents were evaporated and the resultant material partitioned between DCM (2 ml) and saturated aqueous sodium bicarbonate solution (2 ml), gravity filtered through a PTFE cup and evaporated. The resultant material was taken up in DCM and purified on silica eluting with a shallow gradient of 0-5% MeOH/DCM. Fractions containing pure product were combined and evaporated. NMR (400.132 MHz, CDCl₃) 1.05 (m, 2H), 1.23 (m, 4H), 1.38 (s, 9H), 1.49 (d, 6H), 1.63 (m, 2H), 1.91 (m, 1H), 1.99 (m, 4H), 2.10 (m, 2H), 2.50 (s, 3H), 2.65 (m, 2H), 3.73 (m, 2H), 4.01 (m, 2H), 4.83 (m, 1H), 5.17 (m, 1H), 5.50 (m, 1H), 6.65 (d, 1H), 7.24 (s, 1H), 8.13 (d, 1H); MH+ 540.

### Examples 9-13

The following compounds were prepared by the procedure of Example 8 and on the same scale, using the appropriate acid starting material.

| Ex | Compound | NMR (400.132 MHz, CDCl₃) | m/z |
|---|---|---|---|
| 9 | tert-Butyl 4-[2-[[trans-4-[[4-(2- | 1.18 (m, 2H), 1.32 (m, 2H), 1.41 (s, 9H), | 555 |
| | methyl-3-propan-2-yl- | 1.48 (d, 6H), 2.04 (m, 4H), 2.30 (m, 2H), | |
| | imidazol-4-yl)pyrimidin-2- | 2.39 (m, 4H), 2.50 (s, 3H), 2.57 (m, 2H), | |
| | yl]amino]cyclohexyl]carbamoyl]ethyl]piperazine- | 3.37 (m, 4H), 3.72 (m, 2H), 4.84 (m, 1H), | |
| | 1- | 5.50 (m, 1H), 6.64 (d, 1H), 7.23 (s, 1H), | |
| | carboxylate | 7.94 (m, 1H), 8.13 (d, 1H) | |
| 10 | tert-Butyl 4-[2-[[trans-4-[[4-(2- | 1.03 (m, 2H), 1.18 (m, 2H), 1.32 (m, 13H), | 554 |
| | methyl-3-propan-2-yl- | 1.49 (d, 6H), 1.55 (m, 3H), 2.00 (m, 2H), | |
| | imidazol-4-yl)pyrimidin-2- | 2.10 (m, 4H), 2.50 (s, 3H), 2.60 (m, 2H), | |
| | yl]amino]cyclohexyl]carbamoyl]ethyl]piperidine- | 3.72 (m, 2H), 4.00 (m, 2H), 4.84 (m, 1H), | |
| | 1- | 5.16 (m, 1H), 5.51 (m, 1H), 6.64 (d, 1H), | |
| | carboxylate | 7.24 (s, 1H), 8.13 (d, 1H) | |
| 11 | tert-Butyl 4-methyl-4-[[trans- | 1.12 (s, 3H), 1.19 (m, 2H), 1.32 (m, 13H), | 539 |
| | 4-[[4-(2-methyl-3-propan-2-yl- | 1.50 (d, 6H), 1.85 (m, 2H), 1.99 (m, 2H), | |
| | imidazol-4-yl)pyrimidin-2- | 2.11 (m, 2H), 2.51 (s, 3H), 3.18 (m, 2H), | |
| | yl]amino]cyclohexyl]carbamoyl]piperidine- | 3.51 (m, 2H), 3.74 (m, 2H), 4.83 (m, 1H), | |
| | 1- | 5.27 (m, 1H), 5.50 (m, 1H), 6.65 (d, 1H), | |
| | carboxylate | 7.24 (s, 1H), 8.13 (d, 1H) | |
| 12 | tert-Butyl (3R)-3-[[trans-4-[[4- | 1.25 (m, 6H), 1.37 (m, 11H), 1.49 (d, 6H), | 539 |
| | (2-methyl-3-propan-2-yl- | 1.76 (m, 1H), 1.99 (m, 8H), 2.50 (s, 3H), | |
| | imidazol-4-yl)pyrimidin-2- | 3.33 (m, 2H), 3.73 (m, 2H), 4.84 (m, 1H), | |
| | yl]amino]cyclohexyl]carbamoylmethyl]piperidine- | 5.54 (m, 1H), 6.22 (m, 1H), 6.64 (d, 1H), | |
| | 1- | 7.24 (s, 1H), 8.13 (d, 1H) | |
| | carboxylate | | |
| 13 | tert-Butyl (3S)-3-[[trans-4-[[4- | 1.25 (m, 6H), 1.37 (m, 11H), 1.49 (d, 6H), | 539 |
| | (2-methyl-3-propan-2-yl- | 1.76 (m, 1H), 1.99 (m, 8H), 2.50 (s, 3H), | |
| | imidazol-4-yl)pyrimidin-2- | 3.32 (m, 2H), 3.73 (m, 2H), 4.84 (m, 1H), | |
| | yl]amino]cyclohexyl]carbamoylmethyl]piperidine- | 5.54 (m, 1H), 6.22 (m, 1H), 6.64 (d, 1H), | |
| | 1- | 7.24 (s, 1H), 8.12 (d, 1H) | |
| | carboxylate | | |

### Example 14

### N-[trans-4-[[4-(2-Methyl-3-propan-2-yl-imidazol-4-yl)pyrimidin-2-yl]amino]cyclohexyl]-2-(4-piperidyl)acetamide

tert-Butyl 4-[[trans-4-[[4-(2-methyl-3-propan-2-yl-imidazol-4-yl)pyrimidin-2-yl]amino]cyclohexyl]carbamoylmethyl]piperidine-1-carboxylate (Example 8, ˜200 mg, ˜0.38 mmol) was dissolved in DCM (3 ml) and an equal volume of TFA added. The reaction mixture was stirred at ambient temperature for 3 hrs and then added to a 5 g SCX-2 column, pre-wet with MeOH (2 column volumes). The column was with MeOH (2 column volumes) then the product eluted with 2M ammonia in MeOH and the basic eluent evaporated to give the title compound as a glass (52 mg, 31%). NMR (500.133 MHz) 1.29 (m, 2H), 1.39 (m, 4H), 1.51 (d, 6H), 1.80 (m, 2H), 1.98 (m, 3H), 2.06 (m, 2H), 2.48 (s, 3H), 2.86 (m, 2H), 3.23 (m, 2H), 3.57 (m, 1H), 3.71 (m, 1H), 5.60 (m, 1H), 6.73 (d, 1H), 7.28 (s, 1H), 8.16 (d, 1H); MH+ 440.

### Examples 15-19

The following compounds were prepared by the procedure of Example 14 and on the same scale, using the starting materials indicated.

| Ex | Compound | NMR (500.133 MHz) | m/z | SM |
|---|---|---|---|---|
| 15 | 3-(Ethyl-(2- | 1.26 (m, 2H), 1.40 (m, 2H), 1.51 (d, 6H), | 455 | Example 9 |
| | methylaminoethyl)amino)- | 1.89 (m, 2H), 1.97 (m, 2H), 2.20 (m, 2H), | | |
| | N-[trans-4-[[4- | 2.33 (m, 4H), 2.47 (s, 3H), 2.51 (m, 2H), | | |
| | (2-methyl-3-propan-2- | 2.72 (m, 4H), 3.55 (m, 1H), 3.64 (m, 1H), | | |
| | yl-imidazol-4- | 3.72 (m, 1H), 5.63 (m, 1H), 6.44 (d, 1H), | | |
| | yl)pyrimidin-2- | 6.74 (d, 1H), 7.28 (s, 1H), 7.56 (m, 1H), | | |
| | yl]amino]cyclohexyl]propanamide | 8.16 (d, 1H) | | |
| 16 | N-(trans-4-{[4-(1- | 1.02 (m, 2H), 1.28 (m, 3H), 1.38 (m, 2H), | 454 | Example |
| | Isopropyl-2-methyl- | 1.45 (m, 2H), 1.52 (d, 6H), 1.59 (m, 2H), | | 10 |
| | 1H-imidazol-5-yl)pyrimidin- | 1.86 (m, 2H), 1.97 (m, 2H), 2.06 (m, 2H), | | |
| | 2-yl]amino}cyclohexyl)- | 2.45 (m, 5H), 2.94 (m, 2H), 3.55 (m, 1H), | | |
| | 3- | 3.71 (m, 1H), 5.63 (m, 1H), 6.43 (d, 1H), | | |
| | piperidin-4- | 6.74 (d, 1H), 7.22 (m, 1H), 7.28 (s, 1H), | | |
| | ylpropanamide | 8.17 (d, 1H) | | |
| 17 | 4-Methyl-N-[trans-4- | 1.15 (s, 3H), 1.39 (m, 4H), 1.52 (d, 6H), | 440 | Example |
| | [[4-(2-methyl-3- | 1.56 (m, 2H), 1.83 (m, 2H), 2.00 (m, 2H), | | 11 |
| | propan-2-yl-imidazol- | 2.16 (m, 2H), 2.47 (s, 3H), 2.90 (m, 2H), | | |
| | 4-yl)pyrimidin-2-yl]amino]cyclohexyl]piperidine- | 3.12 (m, 2H), 3.64 (m, 1H), 3.71 (m, 1H), | | |
| | 4- | 5.60 (m, 1H), 6.73 (d, 1H), 7.28 (s, 1H), | | |
| | carboxamide | 8.16 (d, 1H) | | |
| 18 | N-[trans-4-[[4-(2- | 1.25 (m, 3H), 1.39 (m, 2H), 1.51 (d, 6H), | 440 | Example |
| | Methyl-3-propan-2-yl- | 1.64 (m, 1H), 1.78 (m, 2H), 1.98 (m, 2H), | | 12 |
| | imidazol-4-yl)pyrimidin- | 2.08 (m, 3H), 2.48 (s, 3H), 2.60 (m, 1H), | | |
| | 2-yl]amino]cyclohexyl]- | 2.76 (m, 1H), 3.21 (m, 2H), 3.57 (m, 1H), | | |
| | 2-[(3R)-3- | 3.71 (m, 1H), 5.60 (m, 1H), 6.73 (d, 1H), | | |
| | piperidyl]acetamide | 7.28 (s, 1H), 8.16 (d, 1H) | | |
| 19 | N-[trans-4-[[4-(2- | 1.25 (m, 3H), 1.39 (m, 2H), 1.51 (d, 6H), | 440 | Example |
| | Methyl-3-propan-2-yl- | 1.64 (m, 1H), 1.78 (m, 2H), 1.99 (m, 2H), | | 13 |
| | imidazol-4-yl)pyrimidin- | 2.09 (m, 3H), 2.48 (s, 3H), 2.61 (m, 1H), | | |
| | 2-yl]amino]cyclohexyl]- | 2.77 (m, 1H), 3.21 (m, 2H), 3.57 (m, 1H), | | |
| | 2-[(3S)-3- | 3.71 (m, 1H), 5.60 (m, 1H), 6.73 (d, 1H), | | |
| | piperidyl]acetamide | 7.28 (s, 1H), 8.16 (d, 1H) | | |

### Example 20

### N-[4-(2-Methyl-3-propan-2-yl-imidazol-4-yl)pyrimidin-2-yl]cyclohexane-1,4-diamine

2-Chloro-4-(2-methyl-3-propan-2-yl-imidazol-4-yl)pyrimidine (Method 2; 1.19 g) was heated with 1,4-diaminocyclohexane (cis/trans isomer mixture; 1.14 g) and triethylamine (0.5 g) in DMA (3.0 ml) at 95° C. for 3 hrs. The mixture was cooled then diluted with 2M aq. sodium carbonate (20 ml) and extracted with DCM (200 ml). The organic layer was dried, filtered then concentrated in vacuo. The residue was purified on silica, eluting with 0-20% MeOH in DCM then 0.5% triethylamine/20% MeOH/DCM to give the title compound as a colourless solid (8:1 mixture of cis/trans isomers) (0.94 g, 54%). NMR (400.13 MHz, CDCl₃) 1.20-2.20 (m, 16H), 2.57 (s, 3H), 2.75-2.95 (m, 1H), 3.75-4.00 (m, 1H), 4.95-5.20 (m, 1H), 5.65 (m, 1H), 6.71 (d, 1H), 7.31 (s, 1H), 8.19 (d, 1H). MH+ 315.

### Example 21

### N-[4-(2-Methyl-3-propan-2-yl-imidazol-4-yl)pyrimidin-2-yl]cyclohexane-1,3-diamine

The title compound (as a 2:1 or 1:2 mixture of cis/trans isomers) was prepared in a similar manner to Example 20 and on a similar scale by using 1,3-diaminocyclohexane (cis/trans isomer mixture)) in place of 1,4-diaminocyclohexane (cis/trans isomer mixture) with 2-chloro-4-(2-methyl-3-propan-2-yl-imidazol-4-yl)pyrimidine (Method 2). NMR (400.13 MHz, CDCl₃) 1.03-1.43 (m, 2H), 1.55 (m, 6H), 1.60-2.30 (m, 8H), 2.56 (s, 3H), 2.84-3.10 (m, 1H), 3.88-4.29 (m, 1H), 5.15 (m, 1H), 5.63 (m, 1H), 6.71 (m, 1H), 7.30 (s, 1H), 8.18 (d, 1H); MH+ 315.

### Example 22

### 3-Dimethylamino-N-[4-[[4-(2-methyl-3-propan-2-yl-imidazol-4-yl)pyrimidin-2-yl]amino]cyclohexyl]propanamide

N-[4-(2-Methyl-3-propan-2-yl-imidazol-4-yl)pyrimidin-2-yl]cyclohexane-1,4-diamine (Example 20; 95 mg) was stirred with 3-(dimethylamino)propionic acid hydrochloride (70 mg) and DIPEA (97 mg) in DMF (1.0 ml) at ambient temperature. HBTU (171 mg) was then added and the solution stirred for 16 hrs. The mixture was purified by RPHPLC to give the title compound as a colourless solid (41 mg, 33%). NMR (400.13 MHz, CDCl₃+D₂O) 1.55 (m, 8H), 1.70 (m, 4H), 1.95 (m, 2H), 2.35 (m, 8H), 2.60 (m, 5H), 4.00 (m, 2H), 5.65 (m, 1H), 6.75 (m, 1H), 7.30 (m, 1H), 8.20 (m, 1H); MH+ 414.

### Examples 23-24

The following compounds were prepared by the procedure of Example 22 and on the same scale, by using the appropriate amine

| Ex | Compound | NMR (400.13 MHz, CDCl₃) | m/z |
|---|---|---|---|
| 23 | 1-Methyl-N-[4-[[4-(2-methyl-3-propan- | 1.20-2.30 (m, 21H), 2.35 (m, | 440 |
| | 2-yl-imidazol-4-yl)pyrimidin-2- | 3H), 2.60 (m, 3H), 2.95 (m, 2H), | |
| | yl]amino]cyclohexyl]piperidine-4- | 3.70-4.10 (m, 2H), 5.65 (m, 1H), | |
| | carboxamide | 6.75 (m, 1H), 7.35 (m, 1H), | |
| | | 8.25 (m, 1H) | |
| 24 | N-[4-[[4-(2-Methyl-3-propan-2-yl- | | 440 |
| | imidazol-4-yl)pyrimidin-2-yl]amino]cyclohexyl]- | | |
| | 3-pyrrolidin-1-yl- | | |
| | propanamide | | |

### Examples 25-27

The following compounds were prepared by the procedure of Example 22 and on the same scale, by using N-[4-(2-methyl-3-propan-2-yl-imidazol-4-yl)pyrimidin-2-yl]cyclohexane-1,3-diamine (Example 21) and the appropriate acid.

| Ex | Compound | NMR (400.13 MHz, CDCl₃) | m/z |
|---|---|---|---|
| 25 | 3-Dimethylamino-N-[3- | (+D₂O) 1.00-1.30 (m, 2H), 1.40-1.65 (m, | 414 |
| | [[4-(2-methyl-3-propan-2- | 8H), 1.65-2.20 (m, 4H), 2.20-2.45 (m, 8H), | |
| | yl-imidazol-4-yl)pyrimidin- | 2.45-2.65 (m, 5H), 3.80-4.25 (m, 2H), | |
| | 2-yl]amino]cyclohexyl]propanamide | 5.45-5.75 (m, 1H), 6.72 (m, 1H), 7.30 (m, 1H), | |
| | | 8.20 (m, 1H) | |
| 26 | 1-Methyl-N-[3-[[4-(2- | (+D₂O) 1.00-1.30 (m, 2H), 1.40-1.65 (m, | 440 |
| | methyl-3-propan-2-yl- | 7H), 1.65-1.95 (m, 6H), 1.95-2.20 (m, 4H), | |
| | imidazol-4-yl)pyrimidin-2- | 2.25-2.45 (m, 4H), 2.58 (s, 3H), | |
| | yl]amino]cyclohexyl]piperidine- | 2.80-3.00 (m, 3H), 3.80-4.25 (m, 2H), 5.45-5.70 (m, | |
| | 4-carboxamide | 1H), 6.72 (m, 1H), 7.30 (m, 1H), 8.20 (m, 1H) | |
| 27 | N-[3-[[4-(2-Methyl-3- | 0.95-1.20 (m, 2H), 1.35-2.15 (m, 16H), | 440 |
| | propan-2-yl-imidazol-4- | 2.35 (m, 2H), 2.55 (m, 7H), 2.70 (m, 2H), | |
| | yl)pyrimidin-2-yl]amino]cyclohexyl]- | 3.80-4.25 (m, 2H), 4.90-5.10 (m, 1H), | |
| | 3-pyrrolidin- | 5.40-5.70 (2m, 1H), 6.70 (2d, 1H), 7.30 (m, 1H), | |
| | 1-yl-propanamide | 8.17 (m, 1H), 8.40-9.10 (m, 1H) | |

### Example 28

### N-[4-[[4-(2-Methyl-3-propan-2-yl-imidazol-4-yl)pyrimidin-2-yl]amino]cyclohexyl]methanesulfonamide

Methanesulfonyl chloride (35 mg) in DCM (1 ml) was added dropwise to a stirred solution of N-[4-(2-methyl-3-propan-2-yl-imidazol-4-yl)pyrimidin-2-yl]cyclohexane-1,4-diamine (Example 20; 95 mg) and triethylamine (46 mg) in DCM (3.0 ml) at ambient temperature. After 1 hr aq. ammonia (25%, 0.5 ml) was added and the mixture was concentrated in vacuo then dissolved in warm DMSO (1 ml) and diluted with water (15 ml). The solid formed was filtered, washed with water and dried in vacuo to give the title compound as a colourless solid (21 mg, 17%). MH+ 393.

### Example 29

### N-[4-[[4-(2-Methyl-3-propan-2-yl-imidazol-4-yl)pyrimidin-2-yl]amino]cyclohexyl]-2-pyrrolidin-1-yl-ethanesulfonamide

N-[4-(2-Methyl-3-propan-2-yl-imidazol-4-yl)pyrimidin-2-yl]cyclohexane-1,4-diamine (Example 20; 95 mg) and DIPEA (117 mg) were dissolved in DCM (3 ml) and cooled to −10° C. 2-Chloroethane sulfonyl chloride (54 mg) in DCM (2 ml) was added dropwise and the reaction was warmed to ambient temperature and stirred for 30 mins. Pyrrolidine (86 mg) was added and the reaction stirred for 16 hrs before concentration in vacuo. The residue was dissolved in DMF (1 ml) and purified by RPHPLC to give the title compound as a colourless solid (43 mg, 30%). NMR (400.13 MHz, CDCl₃) 1.55 (d, 6H), 1.55-1.95 (m, 12H), 2.60 (m, 7H), 3.00 (t, 2H), 3.19 (t, 2H), 3.64 (m, 1H), 3.94 (m, 1H), 5.05 (d, 1H), 5.60 (m, 2H), 6.74 (d, 1H), 7.32 (s, 1H), 8.20 (d, 1H); MH+ 476.

### Example 30

### N-[4-[[4-(2-Methyl-3-propan-2-yl-imidazol-4-yl)pyrimidin-2-yl]amino]cyclohexyl]-3-pyrrolidin-1-yl-propane-1-sulfonamide

N-[4-(2-Methyl-3-propan-2-yl-imidazol-4-yl)pyrimidin-2-yl]cyclohexane-1,4-diamine (Example 20; 95 mg) and DIPEA (117 mg) were dissolved in DCM (3 ml) and cooled to −10° C. 3-Chloropropane sulfonyl chloride (59 mg) in DCM (2 ml) was added dropwise and the reaction warmed to ambient temperature and stirred for 30 mins. The reaction was evaporated in vacuo then pyrrolidine (0.5 ml) was added and the reaction stirred at ambient temperature for 3 days. The reaction was concentrated in vacuo and purified by RPHPLC to give the title compound as a colourless gum (104 mg, 70%). NMR (400.13 MHz, CDCl₃) 1.30-2.25 (m, 20H), 2.55 (m, 9H), 3.12 (t, 2H), 3.30-3.60 (m, 1H), 3.70-4.05 (m, 1H), 4.85-5.40 (m, 2H), 5.60 (m, 1H), 6.73 (m, 1H), 7.30 (m, 1H), 8.20 (m, 1H); MH+ 490.

### Example 31

### N-[3-[[4-(2-Methyl-3-propan-2-yl-imidazol-4-yl)pyrimidin-2-yl]amino]cyclohexyl]methanesulfonamide

N-[4-(2-Methyl-3-propan-2-yl-imidazol-4-yl)pyrimidin-2-yl]cyclohexane-1,3-diamine (Example 21; 95 mg) was stirred with triethylamine (46 mg) in DCM (3 ml) at ambient temperature. Methanesulfonyl chloride (35 mg) in DCM (1.0 ml) was added dropwise and the mixture stirred for 1 hr. Aq. ammonia (25%, 0.5 ml) was added and the mixture diluted with DCM. The organic layer was washed twice with water, dried, filtered and concentrated to give a gum. Trituration with ether gave the title compound as a colourless solid (47 mg, 39%). NMR (400.13 MHz, CDCl₃) 1.05-2.50 (m, 8H), 1.57 (m, 6H), 2.58 (s, 3H), 2.98 (m, 3H), 3.37-3.83 (m, 1H), 3.85-4.27 (m, 1H), 4.63 (m, 1H), 5.00-5.20 (m, 1H), 5.55 (m, 1H), 6.73 (d, 1H), 7.33 (m, 1H), 8.20 (d, 1H); MH+ 393.

### Example 32

### N-[3-[[4-(2-Methyl-3-propan-2-yl-imidazol-4-yl)pyrimidin-2-yl]amino]cyclohexyl]-2-pyrrolidin-1-yl-ethanesulfonamide

The title compound was prepared by the procedure of Example 29 and on the same scale, by using N-[4-(2-methyl-3-propan-2-yl-imidazol-4-yl)pyrimidin-2-yl]cyclohexane-1,3-diamine (Example 21). NMR (400.13 MHz, CDCl₃) 1.05-1.15 (m, 2H), 1.35-2.15 (m, 16H), 2.50 (m, 7H), 2.95 (t, 2H), 3.15 (t, 2H), 3.40-3.80 (m, 1H), 3.85-4.25 (m, 1H), 4.90-5.70 (m, 3H), 6.74 (m, 1H), 7.31 (2s, 1H), 8.20 (m, 1H); MH+ 476.

### Example 33

### N-[3-[[4-(2-Methyl-3-propan-2-yl-imidazol-4-yl)pyrimidin-2-yl]amino]cyclohexyl]-3-pyrrolidin-1-yl-propane-1-sulfonamide

The title compound was prepared by the procedure of Example 30 and on the same scale, by using N-[4-(2-methyl-3-propan-2-yl-imidazol-4-yl)pyrimidin-2-yl]cyclohexane-1,3-diamine (Example 21). NMR (400.13 MHz, CDCl₃) 1.05-1.25 (m, 2H), 1.40-2.20 (m, 18H), 2.50 (m, 9H), 3.10 (m, 2H), 3.35-3.80 (2m, 1H), 3.80-4.30 (2m, 1H), 4.90-5.70 (m, 3H), 6.73 (m, 1H), 7.30 (2s, 1H), 8.20 (m, 1H); MH+ 490.

### Example 34

### 4-[[4-(2-Methyl-3-propan-2-yl-imidazol-4-yl)pyrimidin-2-yl]amino]cyclohexane-1-carboxylic acid lithium salt

(E)-3-Dimethylamino-1-(2-methyl-3-propan-2-yl-imidazol-4-yl)prop-2-en-1-one (Method 24, WO 03/076436; 1.55 g) was refluxed with benzyl cis-4-carbamimidamidocyclohexane-1-carboxylate (Method 8; 1.93 g) in 2-methoxyethanol (10 ml) for 16 hrs. The reaction mixture was cooled then diluted with EtOAc and filtered. The filtrate was washed twice with sat. aq. NaHCO₃, brine, then dried, filtered and concentrated to give an oil. The oil was dissolved in MeOH (20 ml) then stirred with LiOH in water (10 ml) at ambient temperature for 16 hrs. The mixture was concentrated then partitioned between water and EtOAc. The aqueous phase was concentrated then applied to a column of Amberlite XAD2 resin (unfunctionalised polystyrene). Elution with a gradient of 0-10% acetonitrile in water and evaporation of fractions gave the title compound as a yellow foam (2:1 or 1:2 mixture of cis/trans isomers) (1.2 g, 49%). MH+ 344.

Note these conditions and the LiOH hydrolysis lead to epimerisation of the chiral centre next to the acid.

### Example 35

### N-Methyl-4-[[4-(2-methyl-3-propan-2-yl-imidazol-4-yl)pyrimidin-2-yl]amino]cyclohexane-1-carboxamide

4-[[4-(2-Methyl-3-propan-2-yl-imidazol-4-yl)pyrimidin-2-yl]amino]cyclohexane-1-carboxylic acid lithium salt (Example 34; 175 mg) was stirred with methylamine (2M in THF, 0.75 ml) and DIPEA (129 mg) in DMF (1.5 ml) at ambient temperature. HBTU (285 mg) was added and the solution stirred for 16 hrs. The mixture was purified by RPHPLC to give the title compound as a solid (73 mg, 41%). NMR (400.13 MHz, CDCl₃+D₂O) 1.20-2.30 (m, 9H), 1.50 (m, 6H), 2.55 (m, 3H), 2.82 (m, 3H), 3.70-4.10 (m, 1H), 5.60 (m, 2H), 6.71 (m, 1H), 7.30 (m, 1H), 8.18 (m, 1H); MH+ 357.

### Examples 36-38

The following compounds were prepared by the procedure of Example 35 and on the same scale, by using the appropriate amine.

| Ex | Compound | NMR (400.13 MHz, CDCl₃) | m/z |
|---|---|---|---|
| 36 | N-(2-Dimethylaminoethyl)- | (+D₂O) 1.20-2.30 (m, 9H), 1.55 (m, 6H), | 414 |
| | 4-[[4-(2-methyl-3-propan-2- | 2.25 (m, 6H), 2.43 (m, 2H), 2.57 (m, 3H), | |
| | yl-imidazol-4-yl)pyrimidin- | 3.32 (m, 2H), 3.7-4.2 (m, 1H), 5.65 (m, 1H), | |
| | 2-yl]amino]cyclohexane-1- | 6.70 (m, 1H), 7.30 (m, 1H), 8.19 (m, 1H) | |
| | carboxamide | | |
| 37 | (4-Methyl-1,4-diazepan-1- | (+D₂O) 1.20-2.70 (m, 15H), 1.56 (m, 6H), | 440 |
| | yl)-[4-[[4-(2-methyl-3- | 2.36 (m, 3H), 2.56 (m, 3H), 3.60 (m, 4H), | |
| | propan-2-yl-imidazol-4- | 3.7-4.2 (m, 1H), 5.64 (m, 1H), 6.70 (m, 1H), | |
| | yl)pyrimidin-2-yl]amino]cyclohexyl]methanone | 7.31 (m, 1H), 8.20 (m, 1H) | |
| 38 | 4-[[4-(2-Methyl-3-propan- | 1.20-2.40 (m, 12H), 1.57 (m, 6H), | 440 |
| | 2-yl-imidazol-4-yl)pyrimidin- | 2.45-2.65 (m, 9H), 3.3-3.50 (m, 3H), 3.70-4.15 (m, | |
| | 2-yl]amino]-N- | 1H), 4.60-5.35 (m, 1H), 5.65 (m, 1H), | |
| | (2-pyrrolidin-1-ylethyl)cyclohexane- | 6.20 (m, 1H), 6.70 (m, 1H), 7.30 (m, 1H), | |
| | 1-carboxamide | 8.20 (m, 1H) | |

### Example 39

### Methyl 3-[[4-(2-methyl-3-propan-2-yl-imidazol-4-yl)pyrimidin-2-yl]amino]cyclohexane-1-carboxylate

2-Chloro-4-(2-methyl-3-propan-2-yl-imidazol-4-yl)pyrimidine (Method 2; 1.42 g) was heated with methyl 3-aminocyclohexane-1-carboxylate hydrochloride (cis/trans mixture; 2.09 g) and triethylamine (2.2 g) in DMA (5 ml) at 95° C. for 24 hrs. The reaction was cooled, sat. aq. NaHCO₃ (50 ml) added and the aqueous layer extracted with EtOAc (150 ml). The organic layer was washed with water, brine, dried, filtered and concentrated. The residue was triturated with ether-isohexane (1:1) to give a solid, which was filtered off and dried to give the title compound as a colourless solid (˜1:1 mixture of cis/trans isomers) (1.44 g, 67%). NMR (400.13 MHz, CDCl₃) 1.20 (m, 1H), 1.40 (m, 3H), 1.55 (2d, 6H), 1.95 (m, 2H), 2.10 (d, 1H), 2.40 (m, 2H), 2.57 (s, 3H), 3.67 (s, 3H), 3.85 (m, 1H), 5.00 (s, 1H), 5.63 (s, 1H), 6.72 (d, 1H), 7.31 (s, 1H), 8.19 (d, 1H); MH+ 358.

### Example 40

### 3-[[4-(2-Methyl-3-propan-2-yl-imidazol-4-yl)pyrimidin-2-yl]amino]cyclohexane-1-carboxylic acid lithium salt

Methyl 3-[[4-(2-methyl-3-propan-2-yl-imidazol-4-yl)pyrimidin-2-yl]amino]cyclohexane-1-carboxylate (Example 39; 1.43 g) was dissolved in MeOH (10 ml) and stirred with LiOH (0.11 g) in water (5 ml) at ambient temperature for 16 hrs. The mixture was neutralised with solid carbon dioxide pellets then evaporated to dryness to give the title compound as a solid (1.4 g). MH+ 344.

### Examples 41-44

The following compounds were prepared by the procedure of Example 35 and on the same scale, by using 3-[[4-(2-methyl-3-propan-2-yl-imidazol-4-yl)pyrimidin-2-yl]amino]cyclohexane-1-carboxylic acid lithium salt (Example 40) and the appropriate amine.

| Ex | Compound | NMR (400.13 MHz, CDCl₃) | m/z |
|---|---|---|---|
| 41 | N-Methyl-3-[[4-(2-methyl-3- | 1.21 (m, 1H), 1.42 (m, 3H), 1.55 (2d, 6H), | 357 |
| | propan-2-yl-imidazol-4- | 1.92 (m, 2H), 2.12 (m, 1H), 2.28 (m, 2H), 2.56 (s, | |
| | yl)pyrimidin-2-yl]amino]cyclohexane- | 3H), 2.80 (d, 3H), 3.85 (m, 1H), 5.00 (m, 1H), | |
| | 1-carboxamide | 5.50 (m, 1H), 5.56 (m, 1H), 6.71 (d, 1H), | |
| | | 7.30 (s, 1H), 8.19 (d, 1H) | |
| 42 | N-(2-Dimethylaminoethyl)-3- | 1.22 (m, 1H), 1.43 (m, 3H), 1.55 (2d, 6H), | 414 |
| | [[4-(2-methyl-3-propan-2-yl- | 1.91 (m, 2H), 2.12 (m, 1H), 2.21 (s, 6H), 2.29 (m, | |
| | imidazol-4-yl)pyrimidin-2- | 2H), 2.39 (t, 2H), 2.56 (s, 3H), 3.30 (dt, 2H), | |
| | yl]amino]cyclohexane-1- | 3.85 (m, 1H), 5.00 (m, 1H), 5.60 (m, 1H), | |
| | carboxamide | 6.10 (m, 1H), 6.71 (d, 1H), 7.30 (s, 1H), 8.19 (d, | |
| | | 1H) | |
| 43 | (4-Methyl-1,4-diazepan-1- | 1.27 (m, 1H), 1.35-1.65 (m, 9H), 1.9 (m, 4H), | 440 |
| | yl)-[3-[[4-(2-methyl-3- | 2.13 (m, 2H), 2.34 (d, 3H), 2.54 (m, 8H), | |
| | propan-2-yl-imidazol-4- | 3.61 (m, 4H), 3.89 (m, 1H), 5.00 (m, 1H), 5.52 (m, | |
| | yl)pyrimidin-2-yl]amino]cyclohexyl]methanone | 1H), 6.70 (d, 1H), 7.30 (s, 1H), 8.20 (d, 1H) | |
| 44 | 3-[[4-(2-Methyl-3-propan-2- | 1.22 (m, 1H), 1.45 (m, 3H), 1.57 (2d, 6H), | 440 |
| | yl-imidazol-4-yl)pyrimidin- | 1.77 (m, 4H), 1.90 (m, 2H), 2.13 (m, 1H), 2.27 (m, | |
| | 2-yl]amino]-N-(2-pyrrolidin- | 2H), 2.50 (m, 4H), 2.58 (m, 5H), 3.35 (dt, 2H), | |
| | 1-ylethyl)cyclohexane-1- | 3.85 (m, 1H), 5.00 (m, 1H), 5.60 (m, 1H), | |
| | carboxamide | 6.13 (m, 1H), 6.72 (d, 1H), 7.31 (s, 1H), 8.20 (d, | |
| | | 1H) | |

### Example 45

### trans-N-[5-Fluoro-4-(2-methyl-3-propan-2-yl-imidazol-4-yl)pyrimidin-2-yl]cyclohexane-1,4-diamine

2-Chloro-5-fluoro-4-(2-methyl-3-propan-2-yl-imidazol-4-yl)pyrimidine (Method 5; 100 mg, 0.39 mmol) and trans-1,4-diaminocyclohexane (2 ml volume of solid) were heated by microwave irradiation (melt reaction) at 200° C. for 30 mins. The reaction repeated on the same scale then the reactions were combined and purified on silica, eluting with 0-20% MeOH/DCM. Additional purification on silica, eluting with a gradient of 0-10% (2M ammonia in MeOH)/DCM gave the title compound as a colourless foam (245 mg, 94%). NMR (400.132 MHz, CDCl₃) 1.18 (m, 4H), 1.36 (m, 2H), 1.49 (d, 6H), 1.85 (m, 2H), 2.05 (m, 2H), 2.53 (s, 3H), 2.67 (m, 1H), 3.60 (m, 1H), 4.75 (m, 1H), 5.56 (m, 1H), 7.46 (d, 1H), 8.06 (d, 1H); MH+ 333.

### Example 46

### 5-Fluoro-4-(2-methyl-3-propan-2-yl-imidazol-4-yl)-2-[[trans-4-(sulfamoylamino)cyclohexyl]amino]pyrimidine

trans-N-[5-Fluoro-4-(2-methyl-3-propan-2-yl-imidazol-4-yl)pyrimidin-2-yl]cyclohexane-1,4-diamine (Example 45; 126 mg, 0.38 mmol), Sulfamide (546 mg, 5.68 mmol) and dioxane (3 ml) were combined and heated to reflux overnight. The reaction mixture was cooled to ambient temperature and the precipitated solid filtered, washed with MeOH (2×5 ml), water (2×5 ml) and dried to give the title compound as a cream solid (77 mg, 49%). NMR (400.132 MHz) 1.29 (m, 4H), 1.50 (d, 6H), 1.92 (m, 2H), 2.00 (m, 2H), 2.50 (s, 3H), 3.05 (m, 1H), 3.55 (m, 1H), 5.54 (m, 1H), 6.46 (m, 3H), 7.04 (m, 1H), 7.32 (d, 1H), 8.32 (d, 1H); MH+ 412.

### Example 47

### trans-N-[5-Fluoro-4-(2-methyl-3-propan-2-yl-imidazol-4-yl)pyrimidin-2-yl]-N′,N′-dimethyl-cyclohexane-1,4-diamine

The title compound was prepared by the procedure of Example 4 and on the same scale, by using trans-N-[5-fluoro-4-(2-methyl-3-propan-2-yl-imidazol-4-yl)pyrimidin-2-yl]cyclohexane-1,4-diamine (Example 45). NMR (400.132 MHz, CDCl₃) 1.22 (m, 4H), 1.49 (d, 6H), 1.92 (m, 2H), 2.11 (m, 3H), 2.23 (s, 6H), 2.53 (s, 3H), 3.58 (m, 1H), 4.75 (d, 1H), 5.56 (m, 1H), 7.46 (d, 1H), 8.06 (d, 1H); MH+ 361.

### Example 48

### (3R)-3-[[4-(2-Methyl-3-propan-2-yl-imidazol-4-yl)pyrimidin-2-yl]amino]cyclopentane-1-carboxylic acid

Methyl (1S,3R)-3-carbamimidamidocyclopentane-1-carboxylate (Method 7; 1.33 g, 7.18 mmol) and (E)-3-dimethylamino-1-(2-methyl-3-propan-2-yl-imidazol-4-yl)prop-2-en-1-one (Method 24, WO 03/076436; 1.32 g, 5.96 mmol) in 2-methoxyethanol (20 ml) was heated at reflux for 6 hrs. The reaction mixture was cooled, evaporated then partitioned between DCM (100 ml) and water (100 ml). The organic phase was separated, washed with brine, dried, filtered and evaporated to give a yellow oil which was dissolved in THF (10 ml), MeOH (10 ml) and water (5 ml). Lithium hydroxide (284 mg, 11.86 mmol) was added and after stirring for 16 hrs the solvent was evaporated and the residue partitioned between EtOAc (50 ml) and water (50 ml). 2 M HCl was added to the aqueous phase to adjust to pH 4. The aqueous layer was extracted with EtOAc (2×50 ml) and the combined organic extracts were washed with brine, dried, filtered and evaporated to a give yellow solid. The aqueous layer was also evaporated to give a yellow gum. Both products were combined and purified on silica, eluting with a gradient of 0-15% MeOH in DCM, to give the title product as a pale yellow solid (˜1:1 mixture of cis/trans isomers) (563 mg, 29%). NMR (500.133 MHz, 373K) 1.49 (m, 6H), 1.55-1.68 (m, 1H), 1.73-2.24 (m, 5H), 2.46 (s, 3H), 2.71-2.93 (m, 1H), 4.22-4.36 (m, 1H), 5.62 (m, 1H), 6.59-6.68 (m, 1H), 6.74 (m, 1H), 7.27 (m, 1H), 8.17 (d, 1H); MH+ 330.

Note: the position next to the acid racemises during the reaction

### Examples 49-52

The following compounds were prepared by the procedure of Example 35 and on the same scale, by using (3R)-3-[[4-(2-methyl-3-propan-2-yl-imidazol-4-yl)pyrimidin-2-yl]amino]cyclopentane-1-carboxylic acid (Example 48) and the appropriate amine.

| Ex | Compound | NMR (400.13 MHz, DMSO) | m/z |
|---|---|---|---|
| 49 | (3R)—N-Methyl-3-[[4-(2- | 1.50 (m, 6H), 1.50-2.15 (m, 5H), 2.48 (s, 3H), | 343 |
| | methyl-3-propan-2-yl- | 2.55-2.60 (m, 3H), 2.60-2.95 (m, 2H), | |
| | imidazol-4-yl)pyrimidin-2- | 4.05-4.35 (m, 1H), 5.55-5.85 (m, 1H), 6.78 (m, 1H), | |
| | yl]amino]cyclopentane-1- | 7.00-7.30 (m, 1H), 7.37 (m, 1H), 7.60-7.85 (m, 1H), | |
| | carboxamide | 8.18 (m, 1H) | |
| 50 | (3R)—N-(2- | 1.50 (m, 6H), 1.50-2.15 (m, 5H), 2.15 (m, 6H), | 400 |
| | Dimethylaminoethyl)-3-[[4- | 2.30 (m, 2H), 2.48 (s, 3H), 2.65-2.95 (m, 2H), | |
| | (2-methyl-3-propan-2-yl- | 3.15 (m, 2H), 4.05-4.35 (m, 1H), 5.55-5.85 (m, | |
| | imidazol-4-yl)pyrimidin-2- | 1H), 6.78 (m, 1H), 7.00-7.30 (m, 1H), 7.35 (m, | |
| | yl]amino]cyclopentane-1- | 1H), 7.60-7.85 (m, 1H), 8.18 (m, 1H) | |
| | carboxamide | | |
| 51 | (4-Methyl-1,4-diazepan-1- | 1.50 (m, 6H), 1.50-2.20 (m, 7H), 2.30 (m, 3H), | 426 |
| | yl)-[(3R)-3-[[4-(2-methyl- | 2.48 (s, 3H), 2.55-2.95 (m, 2H), 3.10-3.60 (m, | |
| | 3-propan-2-yl-imidazol-4- | 8H), 4.00-4.35 (m, 1H), 5.55-5.85 (m, 1H), | |
| | yl)pyrimidin-2-yl]amino]cyclopentyl]methanone | 6.78 (m, 1H), 7.15 (s, 1H), 7.35 (m, 1H), 8.20 (m, | |
| | | 1H) | |
| 52 | (3R)-3-[[4-(2-Methyl-3- | (CDCl₃) 8.20 (m, 1H), 7.30 (2s, 1H), 6.70 (2d, | 426 |
| | propan-2-yl-imidazol-4- | 1H), 6.15 (m, 1.5H), 5.65 (m, 1H), 5.03 (d, | |
| | yl)pyrimidin-2-yl]amino]- | 0.5H), 4.42 (m, 1H), 3.3-3.5 (m, 3H), 2.57 (m, | |
| | N-(2-pyrrolidin-1-ylethyl)cyclopentane- | 3H), 1.52-2.85 (m, 16H), 1.55 (m, 6H) | |
| | 1-carboxamide | | |

### Example 53

### cis-N-[5-Fluoro-4-(2-methyl-3-propan-2-yl-imidazol-4-al)pyrimidin-2-yl]cyclopentane-1,3-diamine

cis-1,3-Diaminocyclopentane dihydrochloride (Method 9; 2.06 g) was dissolved in MeOH (250 ml) then a solution of KOH (1.34 g) in MeOH (50 ml) was added. The suspension was filtered and the filtrate evaporated to give cis-1,3-diaminocyclopentane (1.2 g; free base) as an oil. 2-Chloro-5-fluoro-4-(2-methyl-3-propan-2-yl-imidazol-4-yl)pyrimidine (Method 5; 2.04 g) was stirred and heated with cis-1,3-diaminocyclopentane (1.2 g) and DIPEA (2.06 g) in DMA (14 ml) at 125° C. for 2 hrs. After which the mixture was concentrated, then diluted with 2M aq. sodium carbonate and extracted with DCM. The organic layer was dried, filtered and purified on silica, eluting with 0-10% MeOH in DCM then 2% triethylamine/15% MeOH/DCM, gave the title compound as a colourless gum (1.62 g, 63% yield). NMR (400.13 MHz, CDCl₃) 1.40 (m, 1H), 1.57 (m, 7H), 1.79 (m, 1H), 1.99 (m, 2H), 2.21 (m, 1H), 2.59 (s, 3H), 3.54 (m, 1H), 4.30 (m, 1H), 5.61 (m, 1H), 5.82 (d, 1H), 7.52 (d, 1H), 8.13 (d, 1H); MH+ 319.

### Examples 54 to 55

The title compound were prepared by the procedure of Example 22 and on the same scale, by using cis-N-[5-fluoro-4-(2-methyl-3-propan-2-yl-imidazol-4-yl)pyrimidin-2-yl]cyclopentane-1,3-diamine (Example 53) and the appropriate acid.

| Ex | Compound | NMR (400.13 MHz, CDCl₃) | m/z |
|---|---|---|---|
| 54 | N-[(cis)-3-[[5-Fluoro-4-(2- | 1.42 (m, 1H), 1.55 (d, 6H), 1.60-1.90 (m, 6H), | 444 |
| | methyl-3-propan-2-yl- | 1.90-2.15 (m, 5H), 2.27 (s, 3H), 2.52 (m, 1H), | |
| | imidazol-4-yl)pyrimidin- | 2.60 (s, 3H), 2.90 (m, 2H), 4.22 (m, 2H), 5.20 (d, | |
| | 2-yl]amino]cyclopentyl]- | 1H), 5.62 (m, 1H), 5.68 (d, 1H), 7.53 (d, 1H), | |
| | 1-methyl-piperidine-4- | 8.14 (d, 1H) | |
| | carboxamide | | |
| 55 | 3-Dimethylamino-N- | 1.45 (m, 1H), 1.55 (d, 6H), 1.65 (m, 2H), 2.06 (m, | 418 |
| | [(cis)-3-[[5-fluoro-4-(2- | 2H), 2.27 (s, 6H), 2.34 (m, 2H), 2.47 (m, 1H), | |
| | methyl-3-propan-2-yl- | 2.54 (m, 2H), 2.60 (s, 3H), 4.22 (m, 2H), 5.22 (d, | |
| | imidazol-4-yl)pyrimidin- | 1H), 5.65 (m, 1H), 7.53 (d, 1H), 8.14 (d, 1H), | |
| | 2-yl]amino]cyclopentyl]propanamide | 8.48 (d, 1H) | |

### Example 56

### N-[cis-3-[[5-Fluoro-4-(2-methyl-3-propan-2-yl-imidazol-4-yl)pyrimidin-2-yl]amino]cyclopentyl]methanesulfonamide

cis-N-[5-Fluoro-4-(2-methyl-3-propan-2-yl-imidazol-4-yl)pyrimidin-2-yl]cyclopentane-1,3-diamine (Example 53; 128 mg) was stirred with triethylamine (61 mg) in DCM (4 ml) at ambient temperature. Methanesulfonyl chloride (46 mg) in DCM (1 ml) was added dropwise and the mixture stirred for 1 hr. Aq. ammonia (25%, 0.5 ml) was added, then the mixture evaporated to dryness and the residue was purified by RPHPLC to give the title compound as a solid (94 mg, 60%). NMR (400.13 MHz, CDCl₃) 1.55 (d, 6H), 1.60 (m, 1H), 1.82 (m, 2H), 2.10 (m, 2H), 2.52 (m, 1H), 2.60 (s, 3H), 2.97 (s, 3H), 3.90 (m, 1H), 4.18 (m, 1H), 5.05 (d, 1H), 5.18 (d, 1H), 5.55 (m, 1H), 7.53 (d, 1H), 8.14 (d, 1H); MH+ 397.

### Examples 57 to 58

The title compound were prepared by the procedure of Example 29 and on the same scale, by using cis-N-[5-fluoro-4-(2-methyl-3-propan-2-yl-imidazol-4-yl)pyrimidin-2-yl]cyclopentane-1,3-diamine (Example 53) and the appropriate amine.

| Ex | Compound | NMR (400.13 MHz, CDCl₃) | m/z |
|---|---|---|---|
| 57 | N-[cis-3-[[5-Fluoro-4-(2- | 1.55 (d, 6H), 1.60 (m, 1H), 1.75 (m, 6H), | 480 |
| | methyl-3-propan-2-yl- | 2.05 (m, 2H), 2.50 (m, 1H), 2.57 (m, 7H), 2.97 (m, | |
| | imidazol-4-yl)pyrimidin-2- | 2H), 3.18 (m, 2H), 3.87 (m, 1H), 4.21 (m, | |
| | yl]amino]cyclopentyl]-2- | 1H), 5.17 (d, 1H), 5.58 (m, 1H), 5.82 (d, 1H), | |
| | pyrrolidin-1-yl- | 7.52 (d, 1H), 8.15 (d, 1H) | |
| | ethanesulfonamide | | |
| 58 | 2-(Dimethylamino)-N-(cis-3- | 1.57 (d, 6H), 1.62 (m, 1H), 1.77 (m, 2H), | 454 |
| | {[5-fluoro-4-(1-isopropyl-2- | 2.08 (m, 2H), 2.27 (s, 6H), 2.49 (m, 1H), 2.60 (s, | |
| | methyl-1H-imidazol-5- | 3H), 2.80 (m, 2H), 3.13 (m, 2H), 3.87 (m, | |
| | yl)pyrimidin-2-yl]amino}cyclopentyl)ethanesulfonamide | 1H), 4.21 (m, 1H), 5.16 (d, 1H), 5.60 (m, | |
| | | 2H), 7.53 (d, 1H), 8.15 (d, 1H) | |

### Preparation of Starting Materials

### Method 1

### 4-(2-Methyl-3-propan-2-yl-imidazol-4-yl)pyrimidin-2-ol

4-(2-Methyl-3-propan-2-yl-imidazol-4-yl)pyrimidin-2-amine (Method 39 WO2003/076436, 5 g, 23 mmol) was dissolved in 70% AcOH—water (145 ml) under an inert atmosphere. Sodium nitrite (5.52 g, 80 mmol) in water (10 ml) was added drop-wise at ambient temperature over a 5 min period giving a mild exotherm. The reaction mixture was heated slowly to 60° C., and held at this temperature for 3 hrs. The reaction mixture was cooled to ambient temperature and neutralised to pH 7 with 40% aq NaOH, extracted with EtOAc (250 ml×5) and the combined extracts dried and evaporated to give the title compound as an off-white solid. (8.2 g, 43%). NMR (400.132 MHz, CDCl₃) 1.51 (d, 6H), 2.03 (s, 3H), 2.54 (s, 3H), 5.93 (m, 1H), 6.60 (d, 1H), 7.57 (m, 2H); MH+ 219.

### Method 2

### 2-Chloro-4-(2-methyl-3-propan-2-yl-imidazol-4-yl)pyrimidine

4-(2-Methyl-3-propan-2-yl-imidazol-4-yl)pyrimidin-2-ol (Method 1, 8.2 g, 29.4 mmol), phosphorous oxychloride (120 ml) and phosphorous pentachloride (6.6 g) were combined and heated at reflux for 18 hrs. Excess phosphorus oxychloride was evaporated the residue dissolved in DCM and stirred in ice and water. The mixture was taken to pH11 by the addition of 40% aqueous sodium hydroxide. The organic and aqueous phases were separated and the organic phased washed with brine, dried and evaporated. The resultant material was dissolved in DCM and chromatographed on silica eluting on a shallow gradient of 0-5% MeOH/DCM. Fractions containing product were combined and evaporated to give the title compound as a pale brown gum. (5.8 g, 84%). NMR (400.132 MHz) 1.53 (d, 6H), 2.50 (s, 3H), 5.27 (m, 1H), 7.72 (s, 1H), 7.79 (d, 1H), 8.62 (d, 1H); MH+ 237.

### Method 3

### 4-Morpholin-4-yl butanoic acid hydrochloride

Ethyl 4-bromobutanoate (67 ml, 0.5 M) was added drop-wise to a solution of morpholine (175 ml, 2 M) in dry toluene (1 l). The reaction mixture was stirred for 4 hrs at 60° C. and then overnight at ambient temperature. The reaction mixture was filtered at 0° C. and the filtrate evaporated. The resultant material was triturated with 60-80 petrol and evaporated to give an orange oil (91.4 g), which was distilled at reduced pressure to give a clear oil (73.2 g) b.p. 90.2° C./3-4 mm Hg. The resultant oil was heated at reflux for 16 hrs in 18% HCl (aq) (1 l). The acid was evaporated leaving a sticky solid which on trituration with ether gave a white solid (75.25 g) which was recrystallized from glacial acetic acid/acetone to give the title compound as a white crystalline solid (56.43 g, 53%) m.p. 181-3° C.

### Method 4

### 5-Fluoro-4-(2-methyl-3-propan-2-yl-imidazol-4-yl)pyrimidin-2-ol acetate

5-Fluoro-4-(2-methyl-3-propan-2-yl-imidazol-4-yl)pyrimidin-2-amine (Method 17 in WO2006/064251; 4 g, 17 mmol) was dissolved in 70% AcOH—water (108 ml) under an inert atmosphere. Sodium nitrite (4.08 g, 59.2 mmol) in water (8 ml) was added dropwise at ambient temperature over 5 mins. The reaction mixture was warmed slowly to 60° C. After 3 hrs the reaction mixture was cooled then neutralised to pH 7 with 40% aq NaOH. The aqueous layer was extracted with EtOAc (6×300 ml), the combined organics dried, filtered and evaporated to give the title compound as a yellow solid (4.07 g, 81%). NMR (400.132 MHz) 1.48 (d, 6H), 1.91 (s, 3H), 2.50 (s, 3H), 5.44 (m, 1H), 7.47 (d, 1H), 8.29 (d, 1H); MH+ 237.

### Method 5

### 2-Chloro-5-fluoro-4-(2-methyl-3-propan-2-yl-imidazol-4-yl)pyrimidine

5-Fluoro-4-(2-methyl-3-propan-2-yl-imidazol-4-yl)pyrimidin-2-ol acetate (Method 4; 4 g, 13.5 mmol) was suspended in phosphorus oxychloride (25 ml) and heated to 90° C. for 3.5 hrs. The reaction mixture was concentrated in vacuo then the residue dissolved in DCM (25 ml) and stirred with ice/water (50 ml). The mixture was cooled in an ice-water bath, neutralised to pH 8 with 40% aq NaOH then water and DCM (50 ml) were added and the organic layer separated. The aqueous layer was extracted with DCM (75 ml) then the combined organics were washed with brine, dried, filtered and evaporated to give a brown oil. Purification by flash chromatography on silica, eluting with 50% EtOAc/iso-hexane, gave the title compound as a yellow oil which crystallised on standing (2.84 g, 83%). NMR (400.132 MHz, CDCl₃) 1.54 (d, 6H), 2.54 (s, 3H), 5.34 (m, 1H), 7.69 (m, 1H), 8.32 (m, 1H); MH+ 255.

### Method 6

### Methyl (1S,3R)-3-aminocyclopentane-1-carboxylate hydrochloride

Thionyl chloride (0.062 ml, 8.57 mmol) was added dropwise over 2-3 mins to cooled (salt/ice bath) anhydrous MeOH (10 ml) under an inert atmosphere. After stirring for 2-3 mins (1S,3R)-3-aminocyclopentane-1-carboxylic acid (997 mg, 7.72 mmol) was added as a solid in a single portion. The reaction mixture was stirred with cooling for 1 hr then for 3 hrs at ambient temperature. The solvent was evaporated and the residue triturated with ether/EtOAc. The solid obtained was collected by filtration and dried to give the title product as a white solid (1.3 g, 94%). NMR (400.132 MHz) 1.62-1.80 (m, 2H), 1.86-1.98 (m, 3H), 2.26 (m, 1H), 2.86 (m, 1H), 3.47 (m, 1H), 3.63 (s, 3H), 8.22 (br s, 3H).

### Method 7

### Methyl (1S,3R)-3-carbamimidamidocyclopentane-1-carboxylate

Methyl (1S,3R)-3-aminocyclopentane-1-carboxylate hydrochloride (Method 6; 1.29 g, 7.18 mmol) was dissolved in acetonitrile (35 ml) and treated with triethylamine (3.0 ml, 21.52 mmol). 1H-Pyrazole-1-carboxamidine hydrochloride (2.1 g, 14.33 mmol) was added and the reaction mixture heated at 70° C. (internal temperature) for 4 hrs. The reaction mixture was allowed to cool overnight and then evaporated to a yellow viscous oil, which was treated with sat. aq. NaHCO₃ (˜50 ml). The mixture was shaken before standing for 30-40 mins. The resultant precipitate produced was collected by suction filtration, washed with water and dried under suction for 2 hrs, before being transferred to a vacuum oven and dried under vacuum, at 55° C., for 3 hrs to give the title compound as a white solid (1.61 g, 100%). NMR (400.132 MHz), 1.44-1.62 (m, 2H), 1.81-1.98 (m, 3H), 2.26 (m, 1H), 2.80 (m, 1H), 3.61 (s, 3H), 3.82 (m, 1H).

### Method 8

### Benzyl cis-4-carbamimidamidocyclohexane-1-carboxylate

The title compound were prepared by the procedure of Method 7 and on the same scale, by using benzyl cis-4-aminocyclohexane-1-carboxylate (Step A, Example 2 in WO06/073589) in place of methyl (1S,3R)-3-aminocyclopentane-1-carboxylate hydrochloride (Method 6). NMR (400.132 MHz) 1.51 (m, 2H), 1.63-1.80 (m, 6H), 2.58 (m, 1H), 3.61 (m, 1H), 5.12 (s, 2H), 7.36 (m, 5H), 7.80 (br. s, 4H).

### Method 9

### cis-Cyclopentane-1,3-diamine dihydrochloride

2,3-Diazabicyclo[2.2.1]heptane dihydrochloride (Tetrahedron Lett., 2002, 43, 5551; 4.06 g) was hydrogenated in EtOH—Water (1:1; 80 ml) in the presence of 10% platinum on activated carbon (2 g) at 40° C. for 7 hrs. The catalyst was filtered through diatomaceous earth and the filtrate evaporated to dryness to give the title compound as a solid (4.1 g, 100%). NMR (400.13 MHz, DMSO+D2O) 1.60 (1H, m), 1.78 (2H, m), 2.02 (2H, m), 2.47 (1H, m), 3.53 (2H, m).

### Example 59

The following illustrate representative pharmaceutical dosage forms containing the compound of formula (I), or a pharmaceutically acceptable salt or in vivo hydrolysable ester thereof (hereafter compound X), for therapeutic or prophylactic use in humans:—

| (a): Tablet I | mg/tablet |
|---|---|
| Compound X | 100 |
| Lactose Ph. Eur | 182.75 |
| Croscarmellose sodium | 12.0 |
| Maize starch paste (5% w/v paste) | 2.25 |
| Magnesium stearate | 3.0 |

| (b): Tablet II | mg/tablet |
|---|---|
| Compound X | 50 |
| Lactose Ph. Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

| (c): Tablet III | mg/tablet |
|---|---|
| Compound X | 1.0 |
| Lactose Ph. Eur | 93.25 |
| Croscarmellose sodium | 4.0 |
| Maize starch paste (5% w/v paste) | 0.75 |
| Magnesium stearate | 1.0 |

| (d): Capsule | mg/capsule |
|---|---|
| Compound X | 10 |
| Lactose Ph. Eur | 488.5 |
| Magnesium stearate | 1.5 |

| (e): Injection I | (50 mg/ml) |
|---|---|
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | to 100% |

| (f): Injection II | 10 mg/ml |
|---|---|
| Compound X | 1.0% w/v |
| Sodium phosphate BP | 3.6% w/v |
| 0.1M Sodium hydroxide solution | 15.0% v/v |
| Water for injection | to 100% |

| (g): Injection III | (1 mg/ml, buffered to pH6) |
|---|---|
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

### Note

The above formulations may be obtained by conventional procedures well known in the pharmaceutical art. The tablets (a)-(c) may be enteric coated by conventional means, for example to provide a coating of cellulose acetate phthalate.

1. A compound of formula (I): 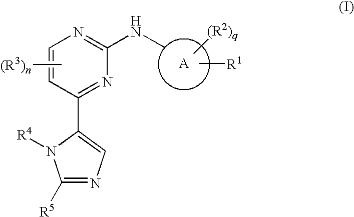
wherein:
Ring A is a 5-7 membered saturated carbocyclic ring wherein 2 atoms of Ring A may optionally be connected by a bridge;
R¹ is selected from carboxy, amino, sulphamoyl, sulphamoylamino, carbamoyl, a group —R⁶-R⁷ or a nitrogen linked 4-7 membered saturated ring which optionally contains an additional nitrogen, oxygen or sulphur atom; wherein said ring may be optionally substituted on carbon by one or more R⁸; and wherein if said ring contains an additional nitrogen atom that nitrogen may be optionally substituted by R⁹;
R² is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆ alkylS(O)ₐ wherein a is 0 to 2, C₁₋₆alkoxycarbonyl, N—(C₁₋₆ alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;
q is 0-4; wherein the values of R² may be the same or different;
R³ is selected from halo, cyano or amino;
n is 0 to 2, wherein the values of R³ may be the same or different;
R⁴ is selected from ethyl, propyl, isopropyl, butyl, iso-butyl, sec-butyl, t-butyl, cyclopropyl, cyclopropylmethyl, 1-cyclopropylethyl, cyclobutylmethyl, cyclopentyl or cyclobutyl; wherein R⁴ may be optionally substituted on carbon by one or more R¹⁴;
R⁵ is selected from methyl, ethyl, isopropyl, fluoromethyl, difluoromethyl, trifluoromethyl, methoxymethyl, cyclopropylmethyl or cyclopropyl;
R⁶ is selected from —O—, —N(R¹⁵)—, —C(O)—, —C(O)O—, —N(R¹⁶)C(O)—, —C(O)N(R¹⁷)—, —N(R¹⁸)C(O)O—, —N(R¹⁹)C(O)N(R²⁰)—, —S(O)ᵣ, —OC(O)N(R²¹)SO₂—, —N(R²²)SO₂N(R²³)—, —SO₂N(R²⁴)—, —N(R²⁵)SO₂—, —C(O)N(R³⁹)SO₂— or —SO₂N(R⁴⁰)C(O)—; wherein R¹⁵, R¹⁶, R¹⁷, R¹⁸, R¹⁹, R²⁰, R²¹, R²², R²³, R²⁴, R²⁵, R³⁹ and R⁴⁰ are independently hydrogen or C₁₋₆alkyl optionally substituted by one or more R²⁶ and r is 0-2;
R⁷ is selected from C₁₋₆alkyl, C₂₋₆alkenyl, C₂₋₆alkynyl, carbocyclyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;
R⁸, R¹², R²⁶ and R²⁷ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²⁹— or heterocyclyl-R³⁰—; wherein R⁸, R¹², R²⁶ and R²⁷ independently of each other may be optionally substituted on carbon by one or more R³¹; and wherein if said heterocyclyl contains an —NH— moiety that nitrogen may be optionally substituted by a group selected from R³²;
R⁹, R¹³, R²⁸ and R³² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹³, R²⁸ and R³² independently of each other may be optionally substituted on carbon by one or more R³³; and
R¹⁰, R¹¹, R²⁹ and R³⁰ are independently selected from a direct bond, —O—, —N(R³⁴)—, —C(O)—, —N(R³⁵)C(O)—, —C(O)N(R³⁶)—, —S(O)ₛ—, —SO₂N(R³⁷)— or —N(R³⁸)SO₂—; wherein R³⁴, R³⁵, R³⁶, R³⁷ and R³⁸ are independently selected from hydrogen or C₁₋₆alkyl and s is 0-2;
R¹⁴ is selected from halo, nitro, cyano, hydroxy, amino, carboxy, carbamoyl, mercapto, sulphamoyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl and C₁₋₆alkylsulphonylamino;
R³¹ and R³³ are independently selected from halo, nitro, cyano, hydroxy, trifluoromethoxy, trifluoromethyl, amino, carboxy, carbamoyl, mercapto, sulphamoyl, methyl, ethyl, cyclopropyl, cyclobutyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt or an in vivo hydrolysable ester thereof.

2. A compound of formula (I), or a pharmaceutically acceptable salt or an in vivo hydrolysable ester thereof, as claimed in claim 1 wherein Ring A is cyclopentyl or cyclohexyl.

3. A compound of formula (I), or a pharmaceutically acceptable salt or an in vivo hydrolysable ester thereof, as claimed in claim 1 wherein R¹ is selected from carboxy, amino, sulphamoylamino or a group —R⁶-R⁷; wherein
R⁶ is selected from —N(R¹⁵)—, —C(O)—, —C(O)O—, —N(R¹⁶)C(O)—, —C(O)N(R¹⁷)—, —N(R¹⁸)C(O)O—, —N(R²²)SO₂N(R²³)— or —N(R²⁵)SO₂—; wherein R¹⁵, R¹⁶, R¹⁷, R¹⁸, R²², R²³ and R²⁵ are independently hydrogen or C₁₋₆alkyl;
R⁷ is selected from C₁₋₆alkyl or heterocyclyl; wherein R⁷ may be optionally substituted on carbon by one or more R²⁷; and wherein if said heterocyclyl contains an —NH— moiety that nitrogen may be optionally substituted by a group selected from R²⁸;
R²⁷ is selected from C₁₋₆alkyl, N,N—(C₁₋₆alkyl)₂amino or heterocyclyl-R³⁰—; and wherein if said heterocyclyl contains an —NH— moiety that nitrogen may be optionally substituted by a group selected from R³²;
R²⁸ and R³² are independently selected from C₁₋₆alkyl and C₁₋₆alkoxycarbonyl; and
R³⁰ is a direct bond.

4. A compound of formula (I), or a pharmaceutically acceptable salt or an in vivo hydrolysable ester thereof, as claimed in claim 1 wherein q is 0.

5. A compound of formula (I), or a pharmaceutically acceptable salt or an in vivo hydrolysable ester thereof, as claimed in claim 1 wherein R³ is halo.

6. A compound of formula (I), or a pharmaceutically acceptable salt or an in vivo hydrolysable ester thereof, as claimed in claim 1 wherein n is 0 or 1.

7. A compound of formula (I), or a pharmaceutically acceptable salt or an in vivo hydrolysable ester thereof, as claimed in claim 1 wherein R⁴ is isopropyl.

8. A compound of formula (I), or a pharmaceutically acceptable salt or an in vivo hydrolysable ester thereof, as claimed in claim 1 wherein R⁵ is methyl.

9. A compound of formula (I): 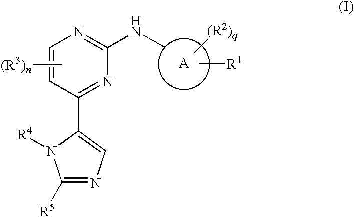
wherein:
Ring A is cyclopentyl or cyclohexyl;
R¹ is selected from amino, carboxy, methoxycarbonyl, sulphamoylamino, N-methylcarbamoyl, N-(2-dimethylaminoethyl)carbamoyl, N-(2-pyrrolidin-1-ylethyl)carbamoyl, N,N-dimethylsulphamoylamino, t-butoxycarbonylamino, mesylamino, dimethylamino, (4-morpholinobutanoyl)amino, 2-(piperidin-4-yl)acetylamino, 2-(N-t-butoxycarbonylpiperidin-4-yl)acetylamino, 3-(piperazin-4-yl)propanoylamino, 3-(1-t-butoxycarbonylpiperazin-4-yl)propanoylamino, 3-(piperidin-4-yl)propanoylamino, 3-(N-t-butoxycarbonylpiperidin-4-yl)propanoylamino, 4-methyl-piperidin-4-ylcarbonylamino, N-t-butoxycarbonyl-4-methyl-piperidin-4-ylcarbonylamino, 2-(pyrrolidin-1-yl)ethylsulphonylamino, 2-(dimethylamino)ethylsulphonylamino, 3-(pyrrolidin-1-yl)propylsulphonylamino, 3-(pyrrolidin-1-yl)propanoylamino, 1-methylhomopiperazin-4-ylcarbonyl, 2-(piperidin-3-yl)acetylamino, 3-(dimethylamino)propanoylamino and 1-methylpiperidin-4-ylcarbonylamino, 2-(N-t-butoxycarbonylpiperidin-3-yl)acetylamino;
q is 0;
R³ is halo;
n is 0 or 1;
R⁴ is isopropyl;
R⁵ is methyl;
or a pharmaceutically acceptable salt or an in vivo hydrolysable ester hereof

10. A compound of formula (I): 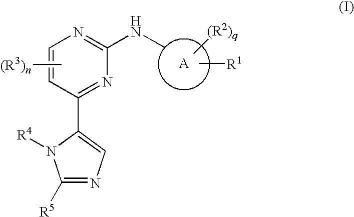
selected from:
5-fluoro-4-(2-methyl-3-propan-2-yl-imidazol-4-yl)-2-[[trans-4-(sulfamoylamino)cyclohexyl]amino]pyrimidine;
N—[cis-3-[[5-fluoro-4-(2-methyl-3-propan-2-yl-imidazol-4-yl)pyrimidin-2-yl]amino]cyclopentyl]methanesulfonamide;
N—[cis-3-[[5-fluoro-4-(2-methyl-3-propan-2-yl-imidazol-4-yl)pyrimidin-2-yl]amino]cyclopentyl]-2-pyrrolidin-1-yl-ethanesulfonamide;
4-(2-methyl-3-propan-2-yl-imidazol-4-yl)-2-[[trans-4-(sulfamoylamino)cyclohexyl]amino]pyrimidine;
trans-N-[5-fluoro-4-(2-methyl-3-propan-2-yl-imidazol-4-yl)pyrimidin-2-yl]cyclohexane-1,4-diamine;
2-(dimethylamino)-N-(cis-3-{[5-fluoro-4-(1-isopropyl-2-methyl-1H-imidazol-5-yl)pyrimidin-2-yl]amino}cyclopentyl)ethanesulfonamide;
N—[trans-[[4-(2-methyl-3-propan-2-yl-imidazol-4-yl)pyrimidin-2-yl]amino]cyclohexyl]methanesulfonamide;
trans-N-[5-fluoro-4-(2-methyl-3-propan-2-yl-imidazol-4-yl)pyrimidin-2-yl]-N′,N′-dimethyl-cyclohexane-1,4-diamine;
N-[(cis)-3-[[5-fluoro-4-(2-methyl-3-propan-2-yl-imidazol-4-yl)pyrimidin-2-yl]amino]cyclopentyl]-1-methyl-piperidine-4-carboxamide; and
N-[trans-4-(2-methyl-3-propan-2-yl-imidazol-4-yl)pyrimidin-2-yl]cyclohexane-1,4-diamine;
or a pharmaceutically acceptable salt or an in vivo hydrolysable ester thereof.

11. A process for preparing a compound of formula (I) or a pharmaceutically acceptable salt or an in vivo hydrolysable ester thereof, as claimed in claim 1, which process comprises:
Process a) reacting a pyrimidine of formula (II): 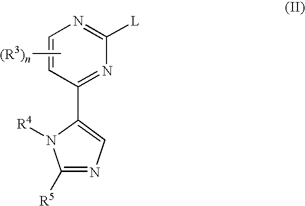
wherein L is a displaceable group; with an amine of formula (III): 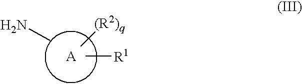
or
Process b) reacting a compound of formula (IV): 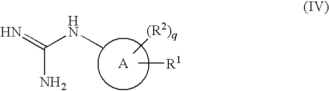
with a compound of formula (V): 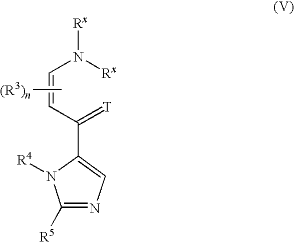
wherein T is O or S; R^{x} may be the same or different and is selected from C₁₋₆alkyl;
or
Process c) reacting a pyrimidine of formula (VI): 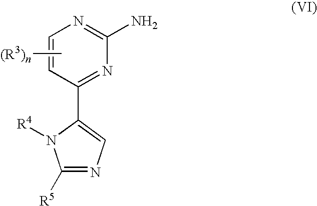
with a compound of formula (VII): 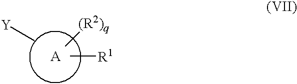
where Y is a displaceable group;
and thereafter optionally:
i) converting a compound of the formula (I) into another compound of the formula (I);
ii) removing any protecting groups;
iii) forming a pharmaceutically acceptable salt or in vivo hydrolysable ester.

12. A pharmaceutical composition which comprises a compound of the formula (I), or a pharmaceutically acceptable salt or in vivo hydrolysable ester thereof, as claimed in any one of claims 1, 9, 10, and a pharmaceutically-acceptable diluent or carrier.

13. A compound of the formula (I), or a pharmaceutically acceptable salt or in vivo hydrolysable ester thereof, as claimed in any one of claims 1, 9, 10, for use as a medicament.

14-18. (canceled)

19. A method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

20. A method of producing a CDK2 inhibitory effect, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

21. A method of treating cancer, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

22. A method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.

23. A method of treating cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as claimed in any one of claims 1, 9, 10.
